(19) United States
(12) Patent Application Publication
(10) Pub. No.: US 2026/0060987 A1
(43) Pub. Date: Mar. 5, 2026

(54) METHODS AND COMPOSITIONS FOR RADIOPROTECTION AND MITIGATION OF RADIATION-INDUCED INJURY USING KINASE INHIBITORS

(71) Applicant: Incyte Corporation, Wilmington, DE (US)

(72) Inventor: Albert Assad, Bound Brook, NJ (US)

(21) Appl. No.: 19/318,283

(22) Filed: Sep. 3, 2025

Related U.S. Application Data
(60) Provisional application No. 63/690,094, filed on Sep. 3, 2024.

(51) Int. Cl. A61K 31/519; A61K 31/4155; A61K 45/06; A61N 5/10; A61P 39/00

(52) U.S. Cl. CPC A61K 31/519; A61K 31/4155; A61K 45/06; A61P 39/00; A61N 5/10

(57) ABSTRACT

The present application provides methods of treating a disease or disorder related to radiation exposure in a patient in need thereof, comprising administering to the patient a therapeutically effective amount of a compound or a pharmaceutically acceptable salt thereof which inhibits kinases such as JAK1, JAK1 and JAK2, or PI3Kδ.

### CROSS-REFERENCE TO RELATED APPLICATIONS

The present application claims the benefit of U.S. Provisional Application No. 63/690,094, filed Sep. 3, 2024, which is incorporated herein by reference in its entirety.

### TECHNICAL FIELD

The present application provides methods for the treatment or prevention of a disease or disorder related to radiation exposure using compounds or pharmaceutically acceptable salts thereof that modulate the activity of kinases such as JAK1, JAK1 and JAK2, or PI3Kδ.

### BACKGROUND

Ionizing radiation, i.e., radiation with energy high enough to have the potential of ionizing molecules in a living body, may cause serious damage and injury to the cells and tissues of living beings. Ionizing radiation damages tissue by direct ionization, which disrupts molecules directly, and also by producing highly reactive free radicals, which attack nearby cells. The net effect is that biological molecules suffer local disruption; this may exceed the body's capacity to repair the damage and may cause mutations in cells currently undergoing replication that lead to diseases. In consequence, dysfunction of many important organs, and even multiple organ failure, can occur, which in turn can eventually lead to death (radiation-induced lethality).

Damaging and harmful effects of radiation can be observed both in the case of acute high dose exposure and in the case of chronic exposure to lower doses. These effects include so-called radiation sickness caused by chronic exposure to the radiation emitting environment, and acute radiation syndrome (poisoning), caused by acute exposure to the internal or external action of a radioactive material or a source of radiation.

Due to extensive use and presence of ionizing radiation and/or radiation sources in many fields of human activity, such as medicine, nuclear power plants, industry, as well as the threat of contamination caused by nuclear/terrorist attacks, the need still exists for drugs which could be effective especially in reducing radiation-induced morbidity and mortality while being nontoxic and safe at concentrations required for effective protection.

### SUMMARY

The present application provides methods of treating or preventing a disease or disorder related to radiation exposure in a patient in need thereof, comprising administering to the patient a therapeutically effective amount of a compound or a pharmaceutically acceptable salt thereof which inhibits JAK1, JAK1 and JAK2, or PI3Kδ.

In some embodiments, the method of treating or preventing a disease or disorder related to radiation exposure in a patient in need thereof comprises administering to the patient a therapeutically effective amount of a compound which inhibits JAK1, JAK1 and JAK2, or PI3Kδ, wherein the compound is:
ruxolitinib;
povorcitinib,
itacitinib;
parsaclisib;
or a pharmaceutically acceptable salt of any of the aforementioned.

In some embodiments, the compound or pharmaceutically acceptable salt thereof is selective for JAK1 and JAK2, which is selective over JAK3 and TYK2.

The present application also provides a compound or a pharmaceutically acceptable salt thereof which inhibits JAK1, JAK1 and JAK2, or PI3Kδ, for use in treating or preventing a disease or disorder related to radiation exposure.

The present application also provides a compound or a pharmaceutically acceptable salt thereof which inhibits JAK1, JAK1 and JAK2, or PI3Kδ, for use in treating or preventing acute radiation syndrome.

The present application further provides use of a compound or a pharmaceutically acceptable salt thereof which inhibits JAK1, JAK1 and JAK2, or PI3Kδ, for preparation of a medicament for use in treatment or prevention of a disease or disorder related to radiation exposure.

The present application further provides use of a compound or a pharmaceutically acceptable salt thereof which inhibits JAK1, JAK1 and JAK2, or PI3Kδ, for preparation of a medicament for use in treatment or prevention of acute radiation syndrome.

The present application further provides a kit comprising: (a) ruxolitinib, povorcitinib, itacitinib, parsaclisib, or a pharmaceutically acceptable salt of any of the aforementioned; and (b) instructions for using the ruxolitinib, povorcitinib, itacitinib, parsaclisib, or a pharmaceutically acceptable salt of any of the aforementioned for treating or preventing acute radiation syndrome of a patient.

### BRIEF DESCRIPTION OF THE FIGURE

FIG. 1 shows preliminary survival data 30 days after radiation exposure of C57Bl/6 mice treated with ruxolitinib, itacitinib, parsaclisib, or povorcitinib as noted in Example B.

FIG. 2 is a bone marrow table demonstrating results upon administration of ruxolitinib, itacitinib, parsaclisib, or povorcitinib to C57Bl/6 mice following radiation exposure resulting in evidence of bone marrow repopulation, supporting a role in hematopoietic recovery as noted in Example C.

FIG. 3 is a Kaplan meir graph demonstrating male survival rates upon administration of ruxolitinib, itacitinib, parsaclisib, or povorcitinib to C57Bl/6 mice as noted in Example D. Administration of ruxolitinib, itacitinib, parsaclisib, or povorcitinib following radiation exposure generally resulted in similar or improved survival rates to vehicle.

### DETAILED DESCRIPTION

The present application provides, inter alia, methods for treating or preventing a disease or disorder related to radiation exposure in a patient in need thereof, comprising administering to the patient a therapeutically effective amount of a kinase inhibitor, or a pharmaceutically acceptable salt thereof.

Anti-inflammatory drugs, such as JAK1 kinase inhibitors, JAK1 and JAK2 kinase inhibitors, or PI3Kδ kinase inhibitors, when used to improve acute radiation survival in humans, have the potential to mitigate the damaging effects of radiation on the body's tissues. This may decrease proinflammatory cytokines among other mechanisms. Acute radiation syndrome can occur when a person is exposed to a high dose of radiation over a short period, leading to severe damage to rapidly dividing cells in the body, particularly in the bone marrow, gastrointestinal tract, and skin.

An anti-inflammatory drug may be effective in treating or preventing a disease or disorder related to radiation exposure as follows:
1. Reduction of Tissue Damage: Radiation causes an inflammatory response that can exacerbate tissue damage. An inflammation drug may help reduce this response, limiting the extent of damage to critical organs and tissues.
2. Preservation of Organ Function: By reducing inflammation, these drugs can help maintain the function of organs that are most susceptible to radiation damage, such as the lungs and gastrointestinal tract. This can be crucial for survival in the acute phase following radiation exposure.
3. Support of Bone Marrow Recovery: Radiation can severely impact bone marrow, leading to decreased production of blood cells and an increased risk of infections and bleeding. Anti-inflammatory drugs might help protect the bone marrow to some extent, aiding in the recovery of blood cell production.
4. Improvement in Overall Survival: By mitigating the harmful effects of inflammation, these drugs can improve overall survival rates by reducing the severity of acute radiation syndrome and improving the body's ability to recover from the radiation injury.

As such, inflammation drugs do not counteract the radiation directly but rather manage the body's response to the injury, thereby improving the chances of survival and recovery.

In some embodiments, the kinase inhibitor is a compound, or a pharmaceutically acceptable salt thereof, which inhibits JAK1, JAK1 and JAK2, or PI3Kδ.

In some embodiments, the compound which inhibits JAK1, JAK1 and JAK2, or PI3Kδ is ruxolitinib, povorcitinib, itacitinib, parsaclisib, or a pharmaceutically acceptable salt of any of the aforementioned.

In some embodiments, the compound or pharmaceutically acceptable salt thereof is selective for JAK1 and JAK2 over JAK3 and TYK2. In some embodiments, the compound or pharmaceutically acceptable salt thereof is (3R)-3-cyclopentyl-3-[4-(7H-pyrrolo[2,3-d]pyrimidin-4-yl)-1H-pyrazol-1-yl]propanenitrile (ruxolitinib), or a pharmaceutically acceptable salt thereof. In some embodiments, the compound or pharmaceutically acceptable salt thereof is ruxolitinib, deuterated forms thereof, stereoisomers thereof, and/or pharmaceutically acceptable salts thereof. Ruxolitinib has an IC₅₀ of less than 10 nM at 1 mM ATP at JAK1 and JAK2. Ruxolitinib can be made by the procedure described in U.S. Pat. No. 7,598,257 (Example 67), which is incorporated herein by reference in its entirety. In some embodiments, the inhibitor of JAK1 and/or JAK2 is ruxolitinib phosphate. The phosphoric acid salt can be made as described in U.S. Pat. No. 8,722,693, which is incorporated herein by reference in its entirety.

In some embodiments, the compound or pharmaceutically acceptable salt thereof is a JAK1 inhibitor. In some embodiments, the compound or pharmaceutically acceptable salt thereof is selective for JAK1 over JAK2, JAK3 and TYK2. In some embodiments, the compound or pharmaceutically acceptable salt thereof is 4-[3-(Cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide (povorcitinib), or a pharmaceutically acceptable salt thereof. In some embodiments, the compound or pharmaceutically acceptable salt thereof is povorcitinib, deuterated forms thereof, stereoisomers thereof, and/or pharmaceutically acceptable salts thereof. Povorcitinib has an IC₅₀ of less than 300 nM at 1 mM ATP at JAK1 with >10 fold selectivity over JAK2. Povorcitinib can be made by the procedure described in U.S. Pat. No. 9,382,231 (Example 7), which is incorporated herein by reference in its entirety. In some embodiments, the inhibitor of JAK1 is povorcitinib phosphate. The phosphoric acid salt can be made as described in U.S. Pat. No. 11,685,731, which is incorporated herein by reference in its entirety.

In other embodiments, the compound or pharmaceutically acceptable salt thereof is {1-{1-[3-Fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl} acetonitrile (itacitinib), or a pharmaceutically acceptable salt thereof. Itacitinib has an IC₅₀ of less than 5 nM at 1 mM ATP at JAK1 with 24.5 fold selectivity over JAK2. In some embodiments, the compound or pharmaceutically acceptable salt thereof is itacitinib, deuterated forms thereof, stereoisomers thereof, and/or pharmaceutically acceptable salts thereof. Itacitinib can be made by the procedure described in U.S. Pat. No. 8,765,734 (Example 1), which is incorporated herein by reference in its entirety. In some embodiments, the inhibitor of JAK1 is itacitinib adipate. The adipic acid salt can be made as described in U.S. Pat. No. 8,987,443, which is incorporated herein by reference in its entirety.

In some embodiments, the compound or pharmaceutically acceptable salt thereof is a PI3Kδ inhibitor. In some embodiments, the compound or pharmaceutically acceptable salt thereof is selective for PI3Kδ over PI3Kα, PI3Kβ, and PI3Kγ. In some embodiments, the compound or pharmaceutically acceptable salt thereof is (R)-4-(3-((S)-1-(4-amino-3-methyl-1H-pyrazolo[3,4-d]pyrimidin-1-yl)ethyl)-5-chloro-2-ethoxy-6-fluorophenyl) pyrrolidin-2-one (parsaclisib), or a pharmaceutically acceptable salt thereof. Parsaclisib has an IC₅₀ of 1 nM at 1 mM ATP at PI3Kδ with 24.5 fold selectivity over other PI3K class I isoforms. In some embodiments, the compound or pharmaceutically acceptable salt thereof is parsaclisib, deuterated forms thereof, stereoisomers thereof, and/or pharmaceutically acceptable salts thereof. Parsaclisib can be made by the procedure described in U.S. Pat. No. 8,765,734 (Examples 345-348), which is incorporated herein by reference in its entirety. In some embodiments, the inhibitor of PI3Kδ is parsaclisib hydrochloride. The hydrochloric acid salt can be made as described in U.S. Pat. No. 10,336,759, which is incorporated herein by reference in its entirety.

In some embodiments, the inhibitors can be an isotopically-labeled compound, or a pharmaceutically acceptable salt thereof. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms, such as —CD₃ being substituted for —CH₃).

One or more constituent atoms of the compounds described herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

Accordingly, in some embodiments, the inhibitor of JAK1, JAK1 and JAK2, or PI3Kδ is a compound wherein one or more hydrogen atoms in the compound are replaced by deuterium atoms, or a pharmaceutically acceptable salt thereof. In some embodiments, the inhibitor of JAK1, JAK1 and JAK2, or PI3Kδ is a compound which is enriched in deuterium.

In some embodiments, the JAK1 and JAK2 inhibitor is ruxolitinib, or a pharmaceutically acceptable salt thereof, wherein one or more hydrogen atoms are replaced by deuterium atoms. In some embodiments, the JAK1 and JAK2 inhibitor is any of the compounds in U.S. Pat. No. 9,249,149 (which is incorporated herein by reference in its entirety), or a pharmaceutically acceptable salt thereof. In some embodiments, the JAK1 and JAK2 inhibitor is CTP-543 (having the structure below) or a pharmaceutically acceptable salt thereof,

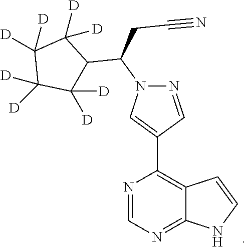 [SMILES: [2H]C1([2H])C([C@@H](CC#N)n2cc(-c3ncnc4[nH]ccc34)cn2)C([2H])([2H])C([2H])([2H])C1([2H])[2H]]

In some embodiments, the JAK1 and JAK2 inhibitor is a compound of Formula I:

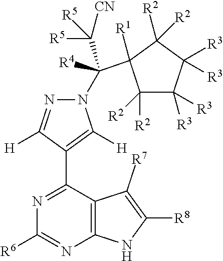

or a pharmaceutically acceptable salt thereot, wherein:
R¹ is selected from H and D;
each R₂ is independently selected from H and D, provided that each R₂ attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R₂ and each R³ are H, R⁴ is H, and each of R⁶, R⁷, and R⁸ is H, then each R⁵ is D.

In some embodiments, the JAK1 and JAK2 inhibitor is a compound of Formula I selected from the following compounds 100-130 in the table below (wherein R⁶, R⁷, and R⁸ are each H), or a pharmaceutically acceptable salt thereof. In some embodiments, the inhibitor of JAK1 and JAK2 inhibitor is a compound of Formula I selected from the following compounds 100-231 in the table below (wherein R⁶, R⁷, and R⁸ are each D), or a pharmaceutically acceptable salt thereof.

| Compound | R¹ | Each R² | Each R³ | R⁴ | Each R⁵ |
|---|---|---|---|---|---|
| 100 | H | H | H | D | H |
| 101 | H | H | H | H | D |
| 102 | H | H | H | D | D |
| 103 | H | H | D | H | H |
| 104 | H | H | D | D | H |
| 105 | H | H | D | H | D |
| 106 | H | H | D | D | D |
| 107 | H | D | H | H | H |
| 108 | H | D | H | D | H |
| 109 | H | D | H | H | D |
| 110 | H | D | H | D | D |
| 111 | H | D | D | H | H |
| 112 | H | D | D | D | H |
| 113 | H | D | D | H | D |
| 114 | H | D | D | D | D |
| 115 | D | H | H | H | H |
| 116 | D | H | H | D | H |
| 117 | D | H | H | H | D |
| 118 | D | H | H | D | D |
| 119 | D | H | D | H | H |
| 120 | D | H | D | D | H |
| 121 | D | H | D | H | D |
| 122 | D | H | D | D | D |
| 123 | D | D | H | H | H |
| 124 | D | D | H | D | H |
| 125 | D | D | H | H | D |
| 126 | D | D | H | D | D |
| 127 | D | D | D | H | H |
| 128 | D | D | D | D | H |
| 129 | D | D | D | H | D |
| 130 | D | D | D | D | D |
| 200 | H | H | H | D | H |
| 201 | H | H | H | H | D |
| 202 | H | H | H | D | D |
| 203 | H | H | D | H | H |
| 204 | H | H | D | D | H |
| 205 | H | H | D | H | D |
| 206 | H | H | D | D | D |
| 207 | H | D | H | H | H |
| 208 | H | D | H | D | H |
| 209 | H | D | H | H | D |
| 210 | H | D | H | D | D |
| 211 | H | D | D | H | H |
| 212 | H | D | D | D | H |
| 213 | H | D | D | H | D |
| 214 | H | D | D | D | D |
| 215 | D | H | H | H | H |
| 216 | D | H | H | D | H |
| 217 | D | H | H | H | D |
| 218 | D | H | H | D | D |
| 219 | D | H | D | H | H |
| 220 | D | H | D | D | H |
| 221 | D | H | D | H | D |
| 222 | D | H | D | D | D |
| 223 | D | D | H | H | H |
| 224 | D | D | H | D | H |
| 225 | D | D | H | H | D |
| 226 | D | D | H | D | D |
| 227 | D | D | D | H | H |
| 228 | D | D | D | D | H |
| 229 | D | D | D | H | D |
| 230 | D | D | D | D | D |
| 231 | H | H | H | H | H |

In some embodiments, the JAK1 and JAK2 inhibitor is baricitinib, wherein one or more hydrogen atoms are replaced by deuterium atoms, or a pharmaceutically acceptable salt thereof. In some embodiments, the inhibitor of JAK1 and JAK2 inhibitor is any of the compounds in U.S. Pat. No. 9,540,367 (which is incorporated herein by reference in its entirety), or a pharmaceutically acceptable salt thereof.

The compounds described herein can be asymmetric (e.g., having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present application. Cis and trans geometric isomers of the compounds of the present application are described and may be isolated as a mixture of isomers or as separated isomeric forms. In some embodiments, the compound has the (R)-configuration. In some embodiments, the compound has the(S)-configuration.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. For example, it will be recognized that the following pyrazole ring may form two tautomers:

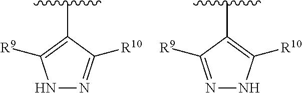

It is intended that the claims cover both tautomers.

All compounds, and pharmaceutically acceptable salts thereof, can be found together with other substances such as water and solvents (e.g. hydrates and solvates) or can be isolated.

In some embodiments, the compounds described herein, or pharmaceutically acceptable salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or pharmaceutically acceptable salts thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expression “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment. In some embodiments, the “subject,” “individual,” or “patient” is a human.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “preventing” refers to blocking the occurrence of disease in a patient who may be predisposed to the disease but does not yet experience or display the pathology or symptomatology of the disease and can also include reducing/decreasing the risk or duration of a disease, or complications associated with a disease such as the need for hospitalization, or death.

As used herein, the term “BID” refers to twice daily dosing. In some embodiments, each of the twice daily doses are separated by at least 6 hours, at least 7 hours, at least 8 hours, or about 12 hours between dosing sessions.

As used herein, “QD” refers to once daily dosing.

In some embodiments, the radiation exposure is via non-therapeutic radiation.

In some embodiments, the disease or disorder is acute radiation syndrome. In some embodiments, the acute radiation syndrome is hematopoietic acute radiation syndrome, radiation-induced coagulopathy, gastrointestinal acute radiation syndrome, cardiovascular acute radiation syndrome, or central nervous system (CNS) acute radiation syndrome or a combination thereof. In some embodiments, the acute radiation syndrome is hematopoietic, gastrointestinal, or a combination thereof. In some embodiments, the acute radiation syndrome is hematopoietic acute radiation syndrome.

In some embodiments, the acute radiation syndrome is gastrointestinal acute radiation syndrome. In some embodiments, the administration reduces radiation-induced gastrointestinal injury, improves crypt survival, or preserves intestinal barrier integrity. In some embodiments, efficacy is observed in the small intestine, colon, mesenteric lymph nodes, or gastrointestinal-associated lymphoid tissue (GALT). In some embodiments, the administration reduces diarrhea, gastrointestinal bleeding, or weight loss associated with radiation exposure.

In some embodiments, the patient is exposed to radiation of about 0.1 Gy (or 10 rads) or greater, about 0.2 Gy (or 20 rads) or greater, about 0.3 Gy (or 30 rads) or greater, about 0.4 Gy (or 40 rads) or greater, about 0.5 Gy (or 50 rads) or greater, about 0.6 Gy (or 60 rads) or greater, about 0.7 Gy (or 70 rads) or greater, about 0.8 Gy (or 80 rads) or greater, about 0.9 Gy (or 90 rads) or greater, about 1 Gy (or 100 rads) or greater, about 2.0 Gy (or 200 rads), about 3.0 Gy (or 300 rads), about 4.0 Gy (or 400 rads) or greater, about 5.0 Gy (or 500 rads), about 6.0 Gy (or 600 rads), about 7.0 Gy (or 700 rads) or greater, about 8.0 Gy (or 800 rads), about 9.0 Gy (or 900 rads), or about 10.0 Gy (or 1000 rads) or greater. In some embodiments, the patient is exposed to radiation of 0.1 Gy or greater. In some embodiments, the patient is exposed to a single dose of total body irradiation of 7.0 Gy.

In some embodiments, the patient is exposed to radiation for at least 1, 2, 5, 10, 30, 60, 80, 120, 180, 240 or 300 seconds.

In some embodiments, the radiation exposure is via therapeutic radiation. In some embodiments, the therapeutic radiation is adjuvant radiation therapy. In some embodiments, the therapeutic radiation is neoadjuvant radiation therapy.

Currently, the tests accepted by regulatory agencies regarding diseases or disorders related to radiation exposure, e.g., acute radiation syndrome, primarily involve survival testing in animals exposed to radiation. In addition to assessing survival (alive/dead), there may be smaller surrogate studies focused on bone marrow sparing, mitigation of GI toxicity, or protection against neurologic toxicity that could eventually augment the overall data.

In some embodiments, administration of the compound or pharmaceutically acceptable salt thereof improves cell survival. In some embodiments, administration of the compound or pharmaceutically acceptable salt thereof improves tissue support. In some embodiments, administration of the compound or pharmaceutically acceptable salt thereof improves bone marrow support. In some embodiments, administration of the compound or pharmaceutically acceptable salt thereof increases the survival rate of the patient. In some embodiments, administration of the compound or pharmaceutically acceptable salt thereof improves progression of hematological repair.

In some embodiments is a method of increasing the survival rate of a patient exposed to radiation, comprising administering to the patient a therapeutically effective amount of a compound or a pharmaceutically acceptable salt thereof which inhibits JAK1, JAK1 and JAK2, or PI3Kδ. In some embodiments, the compound or a pharmaceutically acceptable salt thereof which inhibits JAK1, JAK1 and JAK2, or PI3Kδ is ruxolitinib, povorcitinib, itacitinib, parsaclisib, or a pharmaceutically acceptable salt of any of the aforementioned. In some embodiments, the radiation exposure is via non-therapeutic radiation. In some embodiments, the patient exhibits acute radiation syndrome. In some embodiments, the patient is exposed to radiation of 0.1 Gy or more. In some embodiments, the radiation exposure is via therapeutic radiation. In some embodiments, the therapeutic radiation is adjuvant radiation therapy. In some embodiments, the therapeutic radiation is neoadjuvant radiation therapy. In some embodiments, the compound or pharmaceutically acceptable salt thereof is administered prior to exposure of the patient to radiation. In some embodiments, the compound or pharmaceutically acceptable salt thereof is administered at least 2 days prior to exposure of the patient to radiation. In some embodiments, the compound or pharmaceutically acceptable salt thereof is administered after exposure of the patient to radiation. In some embodiments, the compound or pharmaceutically acceptable salt thereof is first administered within 24 hours after exposure of the patient to radiation.

In some embodiments, the increase or improvement is observed 30 days after radiation exposure. In some embodiments, the increase or improvement is observed at least 30 days after radiation exposure. In some embodiments, the increase or improvement is observed at least 60 days, at least 2 months, at least 3 months, at least 4 months, at least 5 months, at least 6 months, at least 7 months, at least 8 months, at least 9 months, at least 10 months, at least 11 months, at least 12 months, or at least 1 year after radiation exposure. In some embodiments, the increase or improvement is observed at least 60 days after radiation exposure. In some embodiments, the increase or improvement is observed at least 2 months after radiation exposure. In some embodiments, the increase or improvement is observed at least 3 months after radiation exposure. In some embodiments, the increase or improvement is observed at least 6 months after radiation exposure. In some embodiments, the increase or improvement is observed at least 12 months after radiation exposure.

In some embodiments, the compound or pharmaceutically acceptable salt is administered prior to exposure of the patient to radiation. In some embodiments, the compound or pharmaceutically acceptable salt is administered at least 1 day, at least 2 days, at least 3 days, at least 4 days, at least 5 days, at least 6 days, or at least 1 week prior to exposure of the patient to radiation. In some embodiments, the compound or pharmaceutically acceptable salt thereof is administered 1 to 7 days prior to exposure of the patient to radiation. In some embodiments, the compound or pharmaceutically acceptable salt is administered at least 2 days prior to exposure of the patient to radiation. In some embodiments, the compound or pharmaceutically acceptable salt is administered 2 days prior to exposure of the patient to radiation.

In some embodiments, the compound or pharmaceutically acceptable salt is first administered after exposure of the patient to radiation. In some embodiments, the compound or pharmaceutically acceptable salt is first administered within 3, 6, 12, 18, 24, 36, 48, 60 or 72 hours after exposure of the patient to radiation. In some embodiments, the compound or pharmaceutically acceptable salt is first administered within 24 hours after exposure of the patient to radiation.

In some embodiments, the compound or pharmaceutically acceptable salt is administered for at least 1 week, at least 2 weeks, at least 3 weeks, at least 4 weeks, at least 30 days, at least 1 month, at least 2 months, at least 3 months, at least 4 months, at least 5 months, at least 6 months, at least 7 months, at least 8 months, at least 9 months, at least 10 months, at least 11 months, at least 12 months, or at least 1 year. In some embodiments, the compound or pharmaceutically acceptable salt is administered to the patient for at least 30 days. In some embodiments, the compound or pharmaceutically acceptable salt is administered to the patient for at least 60 days. In some embodiments, the compound or pharmaceutically acceptable salt is administered to the patient for at least 2 months. In some embodiments, the compound or pharmaceutically acceptable salt is administered to the patient for at least 3 months. In some embodiments, the compound or pharmaceutically acceptable salt is administered to the patient for at least 6 months. In some embodiments, the compound or pharmaceutically acceptable salt is administered to the patient for at least 12 months.

In some embodiments, the compound or pharmaceutically acceptable salt is first administered within 3, 6, 12, 18, 24, 36, 48, 60 or 72 hours after exposure of the patient to radiation. In some embodiments, the compound or pharmaceutically acceptable salt is first administered within 24 hours after exposure of the patient to radiation.

In some embodiments is a method of treating or preventing acute radiation syndrome, comprising administering to the patient a therapeutically effective amount of a compound which inhibits JAK1, JAK1 and JAK2, or PI3Kδ or a pharmaceutically acceptable salt thereof.

In some embodiments is a method of treating or preventing acute radiation syndrome, comprising administering to the patient a therapeutically effective amount of a compound which inhibits JAK1, JAK1 and JAK2, or PI3Kδ, wherein the compound is:
ruxolitinib;
povorcitinib,
itacitinib;
parsaclisib;
or a pharmaceutically acceptable salt of any of the aforementioned.

In some embodiments is a method of treating or preventing acute radiation syndrome, comprising administering to the patient a therapeutically effective amount of a compound or pharmaceutically acceptable salt thereof that inhibits JAK1 and JAK2. In some embodiments is a method of treating or preventing acute radiation syndrome, comprising administering to the patient a therapeutically effective amount of a compound or pharmaceutically acceptable salt thereof that inhibits JAK1, and is selective for JAK1 and JAK2 over JAK3 and TYK2.

In some embodiments is a method of treating or preventing acute radiation syndrome, comprising administering to the patient a therapeutically effective amount of ruxolitinib, or a pharmaceutically acceptable salt thereof. In some embodiments is a method of treating or preventing acute radiation syndrome, comprising administering to the patient a therapeutically effective amount of ruxolitinib phosphate.

In some embodiments is a method of treating or preventing acute radiation syndrome, comprising administering to the patient a therapeutically effective amount of a compound or pharmaceutically acceptable salt thereof that inhibits JAK1. In some embodiments is a method of treating or preventing acute radiation syndrome, comprising administering to the patient a therapeutically effective amount of a compound or pharmaceutically acceptable salt thereof that inhibits JAK1, and is selective for JAK1 over JAK2, JAK3, and TYK2.

In some embodiments is a method of treating or preventing acute radiation syndrome, comprising administering to the patient a therapeutically effective amount of itacitinib, or a pharmaceutically acceptable salt thereof. In some embodiments is a method of treating or preventing acute radiation syndrome, comprising administering to the patient a therapeutically effective amount of itacitinib adipate.

In some embodiments is a method of treating or preventing acute radiation syndrome, comprising administering to the patient a therapeutically effective amount of povorcitinib, or a pharmaceutically acceptable salt thereof. In some embodiments is a method of treating or preventing acute radiation syndrome, comprising administering to the patient a therapeutically effective amount of povorcitinib phosphate.

In some embodiments is a method of treating or preventing acute radiation syndrome, comprising administering to the patient a therapeutically effective amount of a compound or pharmaceutically acceptable salt thereof that inhibits PI3Kδ. In some embodiments is a method of treating or preventing acute radiation syndrome, comprising administering to the patient a therapeutically effective amount of a compound or pharmaceutically acceptable salt thereof that inhibits PI3Kδ, and is selective for PI3Kδ over PI3Kα, PI3Kβ, and PI3Kγ.

In some embodiments is a method of treating or preventing acute radiation syndrome, comprising administering to the patient a therapeutically effective amount of parsaclisib, or a pharmaceutically acceptable salt thereof. In some embodiments is a method of treating or preventing acute radiation syndrome, comprising administering to the patient a therapeutically effective amount of parsaclisib hydrochloride.

The method is useful in settings including, but not limited to, nuclear disaster, accidental radiation exposure, acute radiation sickness, aviation, spaceflight and space travel, and treatment of military personnel.

In some embodiments, the patient is human.

In some embodiments, the patient was exposed to a nuclear disaster, had an accidental radiation exposure, has acute radiation sickness, was on an aviation flight, was on spaceflight or space travel, or is military personnel undergoing treatment.

In some embodiments, the patient is concurrently receiving radiotherapy. In some embodiments, the radiotherapy is medical therapeutic irradiation.

In some embodiments, the patient is undergoing or has returned from space travel or a space mission. In some embodiments, the patient is an astronaut, space traveler, or spaceflight personnel, and the radiation exposure occurs during orbital, suborbital, or deep-space missions. In some embodiments, the patient was exposed to galactic cosmic rays (GCRs) or solar particle events (SPEs) during the space travel or space mission.

In some embodiments, the patient is being treated for military readiness. In some embodiments, the patient is being treated for radiation exposure obtained during a military operation.

In some embodiments, the patient is a civilian either intentionally or accidentally exposed to a radiation source. In some embodiments, the radiation source is a nuclear reactor, cyclotron, cancer therapy device, nuclear weapon, or radiological weapon.

In some embodiments, the patient is a member of the military exposed to high levels of ionizing radiation, electromagnetic radiation, and radioactive particles. In some embodiments, the exposure is due to radiation powered military facilities or weapons systems.

In some embodiments, the radiation exposure is selected from alpha, beta, gamma, neutron, X-ray radiation, or combinations thereof. In some embodiments, the radiation exposure is in the form of combined or mixed fields of radiation.

In some embodiments, biomarker levels in the patient are assessed prior to administration of therapy. In some embodiments, biomarker levels are assessed during treatment. In some embodiments, dosing of the compound is determined based on the biomarker levels in the patient. In some embodiments, the biomarker is selected from at least one of IL-1B, IL-18, and IL-33.

In some embodiments, the method is supported by survival and hematological recovery data from a validated murine model of hematopoietic acute radiation syndrome (H-ARS), consistent with FDA Animal Rule regulatory requirements.

### Combination Therapies

The methods described herein can further comprise administering one or more additional therapeutic agents. The one or more additional therapeutic agents can be administered to a patient simultaneously or sequentially.

In some embodiments, the additional therapeutic agent is selected from granulocyte-colony stimulating factor (G-CSF), pain medicines, anti-ulcer agents, antidiarrheic, antibiotics, antipyretics, nutritional supplements and antioxidants or combinations thereof.

### Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the compound or pharmaceutically acceptable salt thereof is administered to the patient in a pharmaceutical composition comprising the compound and a pharmaceutically acceptable carrier or excipient.

In some embodiments, the pharmaceutical composition is in a pharmaceutical dosage form. In some embodiments, the pharmaceutical composition is stable at ambient temperature for at least 30 days. In some embodiments, the pharmaceutical composition is in a formulation designed for administration in field-deployable or emergency settings.

In some embodiments, the administration is pulmonary. In some embodiments, the pulmonary administration is in the form of a nebulizer or inhaler.

In some embodiments, the administration is topical. In some embodiments, the topical administration is topical administration to the skin. In some embodiments, the topical administration is in the form of a transdermal patch.

In some embodiments, the administration is parenteral. In some embodiments, the parenteral administration is an intramuscular or subcutaneous injection. In some embodiments, the parenteral administration is in the form of a long acting depot formulation.

In some embodiments, the administration is oral. In some embodiments, the oral administration is in the form of an oral suspension or tablet. In some embodiments, the oral administration is a sustained release formulation. This invention also includes pharmaceutical compositions which contain, as the active ingredient, the compound of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier. In some embodiments, the composition comprises at least one compound described herein, or a pharmaceutically acceptable salt thereof, and at least one component selected from microcrystalline cellulose, lactose monohydrate, hydroxypropyl methylcellulose, and polyethylene oxide. In some embodiments, the composition comprises at least one compound described herein, or a pharmaceutically acceptable salt thereof, and microcrystalline cellulose, lactose monohydrate, and hydroxypropyl methylcellulose. In some embodiments, the composition comprises at least one compound described herein, or a pharmaceutically acceptable salt thereof, and microcrystalline cellulose, lactose monohydrate, and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 1 to about 1,000 mg (1 g), more usually about 1 mg to about 300 mg, of the active ingredient. In some embodiments, each dosage contains about 1 mg of the active ingredient. In some embodiments, each dosage contains about 5 mg of the active ingredient. In some embodiments, each dosage contains about 10 mg of the active ingredient. In some embodiments, each dosage contains about 20 mg of the active ingredient. In some embodiments, each dosage contains about 25 mg of the active ingredient. In some embodiments, each dosage contains about 50 mg of the active ingredient. In some embodiments, each dosage contains about 100 mg of the active ingredient. In some embodiments, each dosage contains about 200 mg of the active ingredient. In some embodiments, each dosage contains about 300 mg of the active ingredient. In some embodiments, each dosage contains about 400 mg of the active ingredient. In some embodiments, each dosage contains about 500 mg of the active ingredient. In some embodiments, each dosage contains about 1000 mg of the active ingredient. In some embodiments, each dosage contains about 5000 mg of the active ingredient.

In some embodiments, the compound or pharmaceutically acceptable salt thereof is administered to the patient at a daily dose in the range of about 1 mg/day to about 8000 mg/day. In some embodiments, the compound or pharmaceutically acceptable salt thereof is administered to the patient at a daily dose in the range of about 1 mg/day to about 2000 mg/day. In some embodiments, the compound or pharmaceutically acceptable salt thereof is administered to the patient at a daily dose in the range of about 1 mg/day to about 1000 mg/day. In some embodiments, the compound or pharmaceutically acceptable salt thereof is administered to the patient at a daily dose in the range of about 100 mg/day to about 1200 mg/day. In some embodiments, the compound or pharmaceutically acceptable salt thereof is administered to the patient at a daily dose in the range of about 5 mg/day to about 25 mg/day. In some embodiments, the compound or pharmaceutically acceptable salt thereof is administered to the patient at a daily dose in the range of about 5 mg/day to about 50 mg/day. In some embodiments, the compound or pharmaceutically acceptable salt thereof is administered to the patient at a daily dose in the range of about 15 mg/day to about 90 mg/day. In some embodiments, the compound or pharmaceutically acceptable salt thereof is administered to the patient at a daily dose in the range of about 5 mg/day to about 45 mg/day. In some embodiments, the compound or pharmaceutically acceptable salt thereof is administered to the patient at a daily dose in the range of about 2 mg/day to about 300 mg/day, about 3 mg/day to about 100 mg/day, about 20 mg/day to about 100 mg/day, about 50 mg/day to about 100 mg/day, about 3 mg/day to about 50 mg/day, about 1 mg/day to about 30 mg/day, about 50 mg/day to about 700 mg/day, about 50 mg/day to about 600 mg/day, or about 100 mg/day to about 2000 mg/day.

In some embodiments, the compound or pharmaceutically acceptable salt thereof is administered to the patient at a daily dose of about 1 mg/day. In some embodiments, the compound or pharmaceutically acceptable salt thereof is administered to the patient at a daily dose of about 2 mg/day. In some embodiments, the compound or pharmaceutically acceptable salt thereof is administered to the patient at a daily dose of about 3 mg/day. In some embodiments, the compound or pharmaceutically acceptable salt thereof is administered to the patient at a daily dose of about 5 mg/day. In some embodiments, the compound or pharmaceutically acceptable salt thereof is administered to the patient at a daily dose of about 10 mg/day. In some embodiments, the compound or pharmaceutically acceptable salt thereof is administered to the patient at a daily dose of about 15 mg/day. In some embodiments, the compound or pharmaceutically acceptable salt thereof is administered to the patient at a daily dose of about 20 mg/day. In some embodiments, the compound or pharmaceutically acceptable salt thereof is administered to the patient at a daily dose of about 25 mg/day. In some embodiments, the compound or pharmaceutically acceptable salt thereof is administered to the patient at a daily dose of about 30 mg/day. In some embodiments, the compound or pharmaceutically acceptable salt thereof is administered to the patient at a daily dose of about 45 mg/day. In some embodiments, the compound or pharmaceutically acceptable salt thereof is administered to the patient at a daily dose of about 50 mg/day. In some embodiments, the compound or pharmaceutically acceptable salt thereof is administered to the patient at a daily dose of about 75 mg/day. In some embodiments, the compound or pharmaceutically acceptable salt thereof is administered to the patient at a daily dose of about 90 mg/day. In some embodiments, the compound or pharmaceutically acceptable salt thereof is administered to the patient at a daily dose of about 100 mg/day. In some embodiments, the compound or pharmaceutically acceptable salt thereof is administered to the patient at a daily dose of about 200 mg/day. In some embodiments, the compound or pharmaceutically acceptable salt thereof is administered to the patient at a daily dose of about 300 mg/day. In some embodiments, the compound or pharmaceutically acceptable salt thereof is administered to the patient at a daily dose of about 400 mg/day. In some embodiments, the compound or pharmaceutically acceptable salt thereof is administered to the patient at a daily dose of about 500 mg/day. In some embodiments, the compound or pharmaceutically acceptable salt thereof is administered to the patient at a daily dose of about 600 mg/day. In some embodiments, the compound or pharmaceutically acceptable salt thereof is administered to the patient at a daily dose of about 750 mg/day. In some embodiments, the compound or pharmaceutically acceptable salt thereof is administered to the patient at a daily dose of about 1000 mg/day. In some embodiments, the compound or pharmaceutically acceptable salt thereof is administered to the patient at a daily dose of about 1200 mg/day. In some embodiments, the compound or pharmaceutically acceptable salt thereof is administered to the patient at a daily dose of about 2000 mg/day. In some embodiments, the compound or pharmaceutically acceptable salt thereof is administered to the patient at a daily dose of about 3000 mg/day. In some embodiments, the compound or pharmaceutically acceptable salt thereof is administered to the patient at a daily dose of about 4000 mg/day. In some embodiments, the compound or pharmaceutically acceptable salt thereof is administered to the patient at a daily dose of about 5000 mg/day. In some embodiments, the compound or pharmaceutically acceptable salt thereof is administered to the patient at a daily dose of about 6000 mg/day. In some embodiments, the compound or pharmaceutically acceptable salt thereof is administered to the patient at a daily dose of about 7000 mg/day. In some embodiments, the compound or pharmaceutically acceptable salt thereof is administered to the patient at a daily dose of about 8000 mg/day.

In some embodiments, the method includes administering to the patient a single dose of the composition. In some embodiments, the method includes administering to the patient multiple doses of the composition. In some embodiments, the method includes administering to the patient from 1 to 4 doses of the composition per day. In some embodiments, the daily dose is administered to the patient once per day. In some embodiments, the daily dose is administered to the patient via twice daily dosing, i.e., a 100 mg daily dose is administered to the patient in two 50 mg doses per day. In some embodiments, the daily dose is administered based on the weight of the free base.

In some embodiments, the compound or pharmaceutically acceptable salt thereof is ruxolitinib, or a pharmaceutically acceptable salt thereof, and is administered to the patient at a daily dose ranging from about 1 mg to about 8000 mg based on the weight of the free base. In some embodiments, the compound or pharmaceutically acceptable salt thereof is ruxolitinib, or a pharmaceutically acceptable salt thereof, and is administered to the patient at a daily dose ranging from about 1 mg to about 1000 mg based on the weight of the free base. In some embodiments, the compound or pharmaceutically acceptable salt thereof is ruxolitinib, or a pharmaceutically acceptable salt thereof, and is administered to the patient at a daily dose ranging from about 100 mg to about 1000 mg based on the weight of the free base. In some embodiments, the compound or pharmaceutically acceptable salt thereof is ruxolitinib, or a pharmaceutically acceptable salt thereof, and is administered to the patient at a daily dose ranging from about 5 mg to about 50 mg based on the weight of the free base. In some embodiments, the compound or pharmaceutically acceptable salt thereof is ruxolitinib, or a pharmaceutically acceptable salt thereof, and is administered to the patient at a daily dose ranging from about 20 mg/kg to about 120 mg/kg based on the weight of the free base. In some embodiments, the compound or pharmaceutically acceptable salt thereof is ruxolitinib, or a pharmaceutically acceptable salt thereof, and is administered to the patient at a daily dose ranging from about 2 mg/kg to about 10 mg/kg based on the weight of the free base. In some embodiments, the ruxolitinib or pharmaceutically acceptable salt thereof is administered QD. In some embodiments the ruxolitinib or pharmaceutically acceptable salt thereof is administered BID. In some embodiments, the compound or pharmaceutically acceptable salt thereof is ruxolitinib, or a pharmaceutically acceptable salt thereof, and is administered to the patient at a daily dose ranging from about 1 mg QD to about 1000 mg QD based on the weight of the free base. In some embodiments, the compound or pharmaceutically acceptable salt thereof is ruxolitinib, or a pharmaceutically acceptable salt thereof, and is administered to the patient at a daily dose ranging from about 5 mg QD to about 25 mg BID based on the weight of the free base.

In some embodiments, the compound or pharmaceutically acceptable salt thereof is itacitinib, or a pharmaceutically acceptable salt thereof, and is administered to the patient at a daily dose ranging from about 1 mg to about 8000 mg based on the weight of the free base. In some embodiments, the compound or pharmaceutically acceptable salt thereof is itacitinib, or a pharmaceutically acceptable salt thereof, and is administered to the patient at a daily dose ranging from about 1 mg to about 2000 mg based on the weight of the free base. In some embodiments, the compound or pharmaceutically acceptable salt thereof is itacitinib, or a pharmaceutically acceptable salt thereof, and is administered to the patient at a daily dose ranging from about 100 mg to about 1200 mg based on the weight of the free base. In some embodiments, the compound or pharmaceutically acceptable salt thereof is itacitinib, or a pharmaceutically acceptable salt thereof, and is administered to the patient at a daily dose ranging from about 100 mg to about 1000 mg based on the weight of the free base. In some embodiments, the compound or pharmaceutically acceptable salt thereof is itacitinib, or a pharmaceutically acceptable salt thereof, and is administered to the patient at a daily dose ranging from about 20 mg/kg to about 120 mg/kg based on the weight of the free base. In some embodiments, the compound or pharmaceutically acceptable salt thereof is itacitinib, or a pharmaceutically acceptable salt thereof, and is administered to the patient at a daily dose ranging from about 2 mg/kg to about 10 mg/kg based on the weight of the free base. In some embodiments, the itacitinib or pharmaceutically acceptable salt thereof is administered QD. In some embodiments the itacitinib or pharmaceutically acceptable salt thereof is administered BID. In some embodiments, the compound or pharmaceutically acceptable salt thereof is itacitinib, or a pharmaceutically acceptable salt thereof, and is administered to the patient at a daily dose ranging from about 1 mg QD to about 2000 mg QD based on the weight of the free base. In some embodiments, the compound or pharmaceutically acceptable salt thereof is itacitinib, or a pharmaceutically acceptable salt thereof, and is administered to the patient at a daily dose ranging from about 100 mg QD to about 1200 mg QD based on the weight of the free base. In some embodiments, the compound or pharmaceutically acceptable salt thereof is itacitinib, or a pharmaceutically acceptable salt thereof, and is administered to the patient at a daily dose ranging from about 100 mg QD to about 600 mg BID based on the weight of the free base.

In some embodiments, the compound or pharmaceutically acceptable salt thereof is povorcitinib, or a pharmaceutically acceptable salt thereof, and is administered to the patient at a daily dose ranging from about 1 mg to about 8000 mg based on the weight of the free base. In some embodiments, the compound or pharmaceutically acceptable salt thereof is povorcitinib, or a pharmaceutically acceptable salt thereof, and is administered to the patient at a daily dose ranging from about 1 mg to about 1000 mg based on the weight of the free base. In some embodiments, the compound or pharmaceutically acceptable salt thereof is povorcitinib, or a pharmaceutically acceptable salt thereof, and is administered to the patient at a daily dose ranging from about 15 mg to about 90 mg based on the weight of the free base. In some embodiments, the compound or pharmaceutically acceptable salt thereof is povorcitinib, or a pharmaceutically acceptable salt thereof, and is administered to the patient at a daily dose ranging from about 10 mg to about 100 mg based on the weight of the free base. In some embodiments, the compound or pharmaceutically acceptable salt thereof is povorcitinib, or a pharmaceutically acceptable salt thereof, and is administered to the patient at a daily dose ranging from about 3 mg/kg to about 10 mg/kg based on the weight of the free base. In some embodiments, the compound or pharmaceutically acceptable salt thereof is povorcitinib, or a pharmaceutically acceptable salt thereof, and is administered to the patient at a daily dose ranging from about 0.2 mg/kg to about 1 mg/kg based on the weight of the free base. In some embodiments, the povorcitinib or pharmaceutically acceptable salt thereof is administered QD. In some embodiments the povorcitinib or pharmaceutically acceptable salt thereof is administered BID. In some embodiments, the compound or pharmaceutically acceptable salt thereof is povorcitinib, or a pharmaceutically acceptable salt thereof, and is administered to the patient at a daily dose ranging from about 1 mg QD to about 1000 mg QD based on the weight of the free base. In some embodiments, the compound or pharmaceutically acceptable salt thereof is povorcitinib, or a pharmaceutically acceptable salt thereof, and is administered to the patient at a daily dose ranging from about 15 mg QD to about 90 mg QD based on the weight of the free base.

In some embodiments, the compound or pharmaceutically acceptable salt thereof is parsaclisib, or a pharmaceutically acceptable salt thereof, and is administered to the patient at a daily dose ranging from about 1 mg to about 8000 mg based on the weight of the free base. In some embodiments, the compound or pharmaceutically acceptable salt thereof is parsaclisib, or a pharmaceutically acceptable salt thereof, and is administered to the patient at a daily dose ranging from about 5 mg to about 45 mg based on the weight of the free base. In some embodiments, the compound or pharmaceutically acceptable salt thereof is parsaclisib, or a pharmaceutically acceptable salt thereof, and is administered to the patient at a daily dose ranging from about 2 mg to about 20 mg based on the weight of the free base. In some embodiments, the compound or pharmaceutically acceptable salt thereof is parsaclisib, or a pharmaceutically acceptable salt thereof, and is administered to the patient at a daily dose ranging from about 0.6 mg/kg to about 6 mg/kg based on the weight of the free base. In some embodiments, the compound or pharmaceutically acceptable salt thereof is parsaclisib, or a pharmaceutically acceptable salt thereof, and is administered to the patient at a daily dose ranging from about 0.04 mg/kg to about 0.4 mg/kg based on the weight of the free base. In some embodiments, the parsaclisib or pharmaceutically acceptable salt thereof is administered QD. In some embodiments the parsaclisib or pharmaceutically acceptable salt thereof is administered BID. In some embodiments, the compound or pharmaceutically acceptable salt thereof is parsaclisib, or a pharmaceutically acceptable salt thereof, and is administered to the patient at a daily dose ranging from about 1 mg QD to about 8000 mg QD based on the weight of the free base. In some embodiments, the compound or pharmaceutically acceptable salt thereof is parsaclisib, or a pharmaceutically acceptable salt thereof, and is administered to the patient at a daily dose ranging from about 5 mg QD to about 45 mg QD based on the weight of the free base.

In some embodiments, the compound or pharmaceutically acceptable salt thereof is ruxolitinib, or a pharmaceutically acceptable salt thereof, and is administered to the patient at a dose ranging from about 1 mg to about 4000 mg twice daily based on the weight of the free base. In some embodiments, the compound or pharmaceutically acceptable salt thereof is ruxolitinib, or a pharmaceutically acceptable salt thereof, and is administered to the patient at a dose ranging from about 10 mg to about 2000 mg twice daily based on the weight of the free base. In some embodiments, the ruxolitinib or pharmaceutically acceptable salt thereof is administered to the patient at a dose ranging from about 100 mg to about 1000 mg twice daily based on the weight of the free base. In some embodiments, the ruxolitinib or pharmaceutically acceptable salt thereof is administered to the patient at a dose ranging from about 50 mg to about 500 mg twice daily based on the weight of the free base. In some embodiments, the ruxolitinib or pharmaceutically acceptable salt thereof is administered to the patient at a dose ranging from about 20 mg to about 750 mg twice daily based on the weight of the free base. In some embodiments, the ruxolitinib or pharmaceutically acceptable salt thereof is administered to the patient at a dose of about 10 mg twice daily based on the weight of the free base. In some embodiments, the ruxolitinib or pharmaceutically acceptable salt thereof is administered to the patient at a dose of about 25 mg twice daily based on the weight of the free base. In some embodiments, the ruxolitinib or pharmaceutically acceptable salt thereof is administered to the patient at a dose of about 50 mg twice daily based on the weight of the free base. In some embodiments, the ruxolitinib or pharmaceutically acceptable salt thereof is administered to the patient at a dose of about 100 mg twice daily based on the weight of the free base. In some embodiments, the ruxolitinib or pharmaceutically acceptable salt thereof is administered to the patient at a dose of about 200 mg twice daily based on the weight of the free base. In some embodiments, the ruxolitinib or pharmaceutically acceptable salt thereof is administered to the patient at a dose of about 300 mg twice daily based on the weight of the free base. In some embodiments, the ruxolitinib or pharmaceutically acceptable salt thereof is administered to the patient at a dose of about 500 mg twice daily based on the weight of the free base. In some embodiments, the ruxolitinib or pharmaceutically acceptable salt thereof is administered to the patient at a dose of about 1000 mg twice daily based on the weight of the free base. In some embodiments, the ruxolitinib or pharmaceutically acceptable salt thereof is administered to the patient at a dose of about 2000 mg twice daily based on the weight of the free base.

In some embodiments, the compound or pharmaceutically acceptable salt thereof is itacitinib, or a pharmaceutically acceptable salt thereof, and is administered to the patient at a dose ranging from about 1 mg to about 4000 mg twice daily based on the weight of the free base. In some embodiments, the compound or pharmaceutically acceptable salt thereof is itacitinib, or a pharmaceutically acceptable salt thereof, and is administered to the patient at a dose ranging from about 10 mg to about 2000 mg twice daily based on the weight of the free base. In some embodiments, the itacitinib or pharmaceutically acceptable salt thereof is administered to the patient at a dose ranging from about 100 mg to about 1000 mg twice daily based on the weight of the free base. In some embodiments, the itacitinib or pharmaceutically acceptable salt thereof is administered to the patient at a dose ranging from about 50 mg to about 500 mg twice daily based on the weight of the free base. In some embodiments, the itacitinib or pharmaceutically acceptable salt thereof is administered to the patient at a dose ranging from about 20 mg to about 750 mg twice daily based on the weight of the free base. In some embodiments, the itacitinib or pharmaceutically acceptable salt thereof is administered to the patient at a dose of about 10 mg twice daily based on the weight of the free base. In some embodiments, the itacitinib or pharmaceutically acceptable salt thereof is administered to the patient at a dose of about 25 mg twice daily based on the weight of the free base. In some embodiments, the itacitinib or pharmaceutically acceptable salt thereof is administered to the patient at a dose of about 50 mg twice daily based on the weight of the free base. In some embodiments, the itacitinib or pharmaceutically acceptable salt thereof is administered to the patient at a dose of about 100 mg twice daily based on the weight of the free base. In some embodiments, the itacitinib or pharmaceutically acceptable salt thereof is administered to the patient at a dose of about 200 mg twice daily based on the weight of the free base. In some embodiments, the itacitinib or pharmaceutically acceptable salt thereof is administered to the patient at a dose of about 300 mg twice daily based on the weight of the free base. In some embodiments, the itacitinib or pharmaceutically acceptable salt thereof is administered to the patient at a dose of about 500 mg twice daily based on the weight of the free base. In some embodiments, the itacitinib or pharmaceutically acceptable salt thereof is administered to the patient at a dose of about 1000 mg twice daily based on the weight of the free base. In some embodiments, the itacitinib or pharmaceutically acceptable salt thereof is administered to the patient at a dose of about 2000 mg twice daily based on the weight of the free base.

In some embodiments, the compound or pharmaceutically acceptable salt thereof is parsaclisib, or a pharmaceutically acceptable salt thereof, and is administered to the patient at a dose ranging from about 1 mg to about 4000 mg twice daily based on the weight of the free base. In some embodiments, the compound or pharmaceutically acceptable salt thereof is parsaclisib, or a pharmaceutically acceptable salt thereof, and is administered to the patient at a dose ranging from about 1 mg to about 500 mg twice daily based on the weight of the free base. In some embodiments, the parsaclisib or pharmaceutically acceptable salt thereof is administered to the patient at a dose ranging from about 3 mg to about 250 mg twice daily based on the weight of the free base. In some embodiments, the parsaclisib or pharmaceutically acceptable salt thereof is administered to the patient at a dose ranging from about 5 mg to about 50 mg twice daily based on the weight of the free base. In some embodiments, the parsaclisib or pharmaceutically acceptable salt thereof is administered to the patient at a dose ranging from about 1 mg to about 10 mg twice daily based on the weight of the free base. In some embodiments, the parsaclisib or pharmaceutically acceptable salt thereof is administered to the patient at a dose of about 1 mg twice daily based on the weight of the free base. In some embodiments, the parsaclisib or pharmaceutically acceptable salt thereof is administered to the patient at a dose of about 3 mg twice daily based on the weight of the free base. In some embodiments, the parsaclisib or pharmaceutically acceptable salt thereof is administered to the patient at a dose of about 10 mg twice daily based on the weight of the free base. In some embodiments, the parsaclisib or pharmaceutically acceptable salt thereof is administered to the patient at a dose of about 25 mg twice daily based on the weight of the free base. In some embodiments, the parsaclisib or pharmaceutically acceptable salt thereof is administered to the patient at a dose of about 50 mg twice daily based on the weight of the free base. In some embodiments, the parsaclisib or pharmaceutically acceptable salt thereof is administered to the patient at a dose of about 100 mg twice daily based on the weight of the free base. In some embodiments, the parsaclisib or pharmaceutically acceptable salt thereof is administered to the patient at a dose of about 250 mg twice daily based on the weight of the free base. In some embodiments, the parsaclisib or pharmaceutically acceptable salt thereof is administered to the patient at a dose of about 500 mg twice daily based on the weight of the free base. In some embodiments, the parsaclisib or pharmaceutically acceptable salt thereof is administered to the patient at a dose of about 1000 mg twice daily based on the weight of the free base.

In some embodiments, the compound or pharmaceutically acceptable salt thereof is povorcitinib, or a pharmaceutically acceptable salt thereof, and is administered to the patient at a dose ranging from about 1 mg to about 4000 mg once daily based on the weight of the free base. In some embodiments, the compound or pharmaceutically acceptable salt thereof is povorcitinib, or a pharmaceutically acceptable salt thereof, and is administered to the patient at a dose ranging from about 1 mg to about 500 mg once daily based on the weight of the free base. In some embodiments, the povorcitinib or pharmaceutically acceptable salt thereof is administered to the patient at a dose ranging from about 3 mg to about 250 mg once daily based on the weight of the free base. In some embodiments, the povorcitinib or pharmaceutically acceptable salt thereof is administered to the patient at a dose ranging from about 10 mg to about 100 mg once daily based on the weight of the free base. In some embodiments, the povorcitinib or pharmaceutically acceptable salt thereof is administered to the patient at a dose ranging from about 5 mg to about 50 mg once daily based on the weight of the free base. In some embodiments, the povorcitinib or pharmaceutically acceptable salt thereof is administered to the patient at a dose of about 1 mg once daily based on the weight of the free base. In some embodiments, the povorcitinib or pharmaceutically acceptable salt thereof is administered to the patient at a dose of about 3 mg once daily based on the weight of the free base. In some embodiments, the povorcitinib or pharmaceutically acceptable salt thereof is administered to the patient at a dose of about 10 mg once daily based on the weight of the free base. In some embodiments, the povorcitinib or pharmaceutically acceptable salt thereof is administered to the patient at a dose of about 25 mg once daily based on the weight of the free base. In some embodiments, the povorcitinib or pharmaceutically acceptable salt thereof is administered to the patient at a dose of about 50 mg once daily based on the weight of the free base. In some embodiments, the povorcitinib or pharmaceutically acceptable salt thereof is administered to the patient at a dose of about 100 mg once daily based on the weight of the free base. In some embodiments, the povorcitinib or pharmaceutically acceptable salt thereof is administered to the patient at a dose of about 250 mg once daily based on the weight of the free base. In some embodiments, the povorcitinib or pharmaceutically acceptable salt thereof is administered to the patient at a dose of about 500 mg once daily based on the weight of the free base. In some embodiments, the povorcitinib or pharmaceutically acceptable salt thereof is administered to the patient at a dose of about 1000 mg once daily based on the weight of the free base.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. In some embodiments, the dose range is from about 0.02 mg/kg to about 20 mg/kg, about 0.05 mg/kg to about 10 mg/kg, 0.1 mg/kg to about 10 mg/kg, 0.2 mg/kg to about 8 mg/kg, 0.5 mg/kg to about 5 mg/kg, 1 mg/kg to about 5 mg/kg, or 2 mg/kg to about 3 mg/kg of body weight per day. In some embodiments, the dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg of body weight per day.

In some embodiments, the compound or pharmaceutically acceptable salt thereof is ruxolitinib, or a pharmaceutically acceptable salt thereof, and is administered to the patient at a dose ranging from about 0.02 mg/kg to about 100 mg/kg twice daily based on the weight of the free base. In some embodiments, the ruxolitinib or pharmaceutically acceptable salt thereof is administered to the patient at a dose ranging from about 10 mg/kg to about 60 mg/kg twice daily based on the weight of the free base. In some embodiments, the ruxolitinib or pharmaceutically acceptable salt thereof is administered to the patient at a dose ranging from about 1 mg/kg to about 10 mg/kg twice daily based on the weight of the free base. In some embodiments, the ruxolitinib or pharmaceutically acceptable salt thereof is administered to the patient at a dose of about 1 mg/kg twice daily based on the weight of the free base. In some embodiments, the ruxolitinib or pharmaceutically acceptable salt thereof is administered to the patient at a dose of about 5 mg/kg twice daily based on the weight of the free base. In some embodiments, the ruxolitinib or pharmaceutically acceptable salt thereof is administered to the patient at a dose of about 10 mg/kg twice daily based on the weight of the free base. In some embodiments, the ruxolitinib or pharmaceutically acceptable salt thereof is administered to the patient at a dose of about 20 mg/kg mg twice daily based on the weight of the free base. In some embodiments, the ruxolitinib or pharmaceutically acceptable salt thereof is administered to the patient at a dose of about 25 mg/kg twice daily based on the weight of the free base. In some embodiments, the ruxolitinib or pharmaceutically acceptable salt thereof is administered to the patient at a dose of about 50 mg/kg twice daily based on the weight of the free base. In some embodiments, the ruxolitinib or pharmaceutically acceptable salt thereof is administered to the patient at a dose of about 60 mg/kg twice daily based on the weight of the free base. In some embodiments, the ruxolitinib or pharmaceutically acceptable salt thereof is administered to the patient at a dose of about 75 mg/kg twice daily based on the weight of the free base. In some embodiments, the ruxolitinib or pharmaceutically acceptable salt thereof is administered to the patient at a dose of about 100 mg/kg twice daily based on the weight of the free base.

In some embodiments, the compound or pharmaceutically acceptable salt thereof is itacitinib, or a pharmaceutically acceptable salt thereof, and is administered to the patient at a dose ranging from about 0.02 mg/kg to about 100 mg/kg twice daily based on the weight of the free base. In some embodiments, the itacitinib or pharmaceutically acceptable salt thereof is administered to the patient at a dose ranging from about 10 mg/kg to about 60 mg/kg twice daily based on the weight of the free base. In some embodiments, the itacitinib or pharmaceutically acceptable salt thereof is administered to the patient at a dose ranging from about 1 mg/kg to about 10 mg/kg twice daily based on the weight of the free base. In some embodiments, the itacitinib or pharmaceutically acceptable salt thereof is administered to the patient at a dose of about 1 mg/kg twice daily based on the weight of the free base. In some embodiments, the itacitinib or pharmaceutically acceptable salt thereof is administered to the patient at a dose of about 5 mg/kg twice daily based on the weight of the free base. In some embodiments, the itacitinib or pharmaceutically acceptable salt thereof is administered to the patient at a dose of about 10 mg/kg twice daily based on the weight of the free base. In some embodiments, the itacitinib or pharmaceutically acceptable salt thereof is administered to the patient at a dose of about 20 mg/kg mg twice daily based on the weight of the free base. In some embodiments, the itacitinib or pharmaceutically acceptable salt thereof is administered to the patient at a dose of about 25 mg/kg twice daily based on the weight of the free base. In some embodiments, the itacitinib or pharmaceutically acceptable salt thereof is administered to the patient at a dose of about 50 mg/kg twice daily based on the weight of the free base. In some embodiments, the itacitinib or pharmaceutically acceptable salt thereof is administered to the patient at a dose of about 60 mg/kg twice daily based on the weight of the free base. In some embodiments, the itacitinib or pharmaceutically acceptable salt thereof is administered to the patient at a dose of about 75 mg/kg twice daily based on the weight of the free base. In some embodiments, the itacitinib or pharmaceutically acceptable salt thereof is administered to the patient at a dose of about 100 mg/kg twice daily based on the weight of the free base.

In some embodiments, the compound or pharmaceutically acceptable salt thereof is parsaclisib, or a pharmaceutically acceptable salt thereof, and is administered to the patient at a dose ranging from about 0.02 mg/kg to about 100 mg/kg twice daily based on the weight of the free base. In some embodiments, the parsaclisib or pharmaceutically acceptable salt thereof is administered to the patient at a dose ranging from about 0.3 mg/kg to about 10 mg/kg twice daily based on the weight of the free base. In some embodiments, the parsaclisib or pharmaceutically acceptable salt thereof is administered to the patient at a dose ranging from about 0.02 mg/kg to about 1 mg/kg twice daily based on the weight of the free base. In some embodiments, the parsaclisib or pharmaceutically acceptable salt thereof is administered to the patient at a dose ranging from about 0.02 mg/kg to about 0.2 mg/kg twice daily based on the weight of the free base. In some embodiments, the parsaclisib or pharmaceutically acceptable salt thereof is administered to the patient at a dose of about 0.02 mg/kg twice daily based on the weight of the free base. In some embodiments, the parsaclisib or pharmaceutically acceptable salt thereof is administered to the patient at a dose of about 0.1 mg/kg twice daily based on the weight of the free base. In some embodiments, the parsaclisib or pharmaceutically acceptable salt thereof is administered to the patient at a dose of about 0.5 mg/kg twice daily based on the weight of the free base. In some embodiments, the parsaclisib or pharmaceutically acceptable salt thereof is administered to the patient at a dose of about 1 mg/kg mg twice daily based on the weight of the free base. In some embodiments, the parsaclisib or pharmaceutically acceptable salt thereof is administered to the patient at a dose of about 2 mg/kg twice daily based on the weight of the free base. In some embodiments, the parsaclisib or pharmaceutically acceptable salt thereof is administered to the patient at a dose of about 3 mg/kg twice daily based on the weight of the free base. In some embodiments, the parsaclisib or pharmaceutically acceptable salt thereof is administered to the patient at a dose of about 5 mg/kg twice daily based on the weight of the free base. In some embodiments, the parsaclisib or pharmaceutically acceptable salt thereof is administered to the patient at a dose of about 10 mg/kg twice daily based on the weight of the free base. In some embodiments, the parsaclisib or pharmaceutically acceptable salt thereof is administered to the patient at a dose of about 25 mg/kg twice daily based on the weight of the free base.

In some embodiments, the compound or pharmaceutically acceptable salt thereof is povorcitinib, or a pharmaceutically acceptable salt thereof, and is administered to the patient at a dose ranging from about 0.1 mg/kg to about 100 mg/kg once daily based on the weight of the free base. In some embodiments, the povorcitinib or pharmaceutically acceptable salt thereof is administered to the patient at a dose ranging from about 3 mg/kg to about 10 mg/kg once daily based on the weight of the free base. In some embodiments, the povorcitinib or pharmaceutically acceptable salt thereof is administered to the patient at a dose ranging from about 0.2 mg/kg to about 1 mg/kg once daily based on the weight of the free base. In some embodiments, the povorcitinib or pharmaceutically acceptable salt thereof is administered to the patient at a dose of about 0.1 mg/kg once daily based on the weight of the free base. In some embodiments, the povorcitinib or pharmaceutically acceptable salt thereof is administered to the patient at a dose of about 0.2 mg/kg once daily based on the weight of the free base. In some embodiments, the povorcitinib or pharmaceutically acceptable salt thereof is administered to the patient at a dose of about 0.5 mg/kg once daily based on the weight of the free base. In some embodiments, the povorcitinib or pharmaceutically acceptable salt thereof is administered to the patient at a dose of about 1 mg/kg mg once daily based on the weight of the free base. In some embodiments, the povorcitinib or pharmaceutically acceptable salt thereof is administered to the patient at a dose of about 2 mg/kg once daily based on the weight of the free base. In some embodiments, the povorcitinib or pharmaceutically acceptable salt thereof is administered to the patient at a dose of about 3 mg/kg once daily based on the weight of the free base. In some embodiments, the povorcitinib or pharmaceutically acceptable salt thereof is administered to the patient at a dose of about 5 mg/kg once daily based on the weight of the free base. In some embodiments, the povorcitinib or pharmaceutically acceptable salt thereof is administered to the patient at a dose of about 10 mg/kg once daily based on the weight of the free base. In some embodiments, the povorcitinib or pharmaceutically acceptable salt thereof is administered to the patient at a dose of about 20 mg/kg once daily based on the weight of the free base.

The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt. % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents, examples of which are listed herein above.

### Kits

The present application also includes pharmaceutical kits useful, for example, in the treatment and/or prevention of diseases or disorders related to radiation exposure, such as acute radiation syndrome, which include one or more containers containing a pharmaceutical composition comprising a therapeutically effective amount of a compound or pharmaceutically acceptable salt described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

In some embodiments, the kit comprises: (a) a compound which inhibits JAK1, JAK1 and JAK2, or PI3Kδ, or a pharmaceutically acceptable salt thereof; and (b) instructions for using the compound which inhibits JAK1, JAK1 and JAK2, or PI3Kδ, or a pharmaceutically acceptable salt thereof for treating or preventing a disease or disorder related to radiation exposure of a patient. In some embodiments, the kit comprises: (a) ruxolitinib, povorcitinib, itacitinib, parsaclisib, or a pharmaceutically acceptable salt of any of the aforementioned; and (b) instructions for using the ruxolitinib, povorcitinib, itacitinib, parsaclisib, or a pharmaceutically acceptable salt of any of the aforementioned for treating or preventing acute radiation syndrome of a patient.

In some embodiments, the kit requires no refrigeration. In some embodiments, the kit is a filed deployable kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example A: Proof of Concept Study in a Mouse Irradiation Model

### 1. Objective

The objective of the study is to determine the efficacy, specifically the survival benefit and progression of hematological repair of ruxolitinib, itacitinib, parsaclisib, and povorcitinib at 30 days following lethal total body irradiation in a mouse model of hematopoietic acute radiation syndrome (hARS).

### 2. Justification

The in vivo irradiation mouse model developed at the Lovelace Biomedical will be used in the studies focused on treatment of hematopoietic acute radiation syndrome (H-ARS). Using this model, the current study aims to compare the survival in ten experimental groups (ten animals each): Irradiation Control group, three ruxolitinib groups (treated with prophylactic, low, and high doses, respectively), two itacitinib groups (low and high doses, respectively), two parsaclisib groups (low and high doses, respectively), and two povorcitinib groups (low and high doses, respectively). Additionally, Sham Control group (ten animals) will be used as a negative control (no irradiation or medication treatment).

C57Bl/6 mice will be chosen based on previous studies conducted both at Lovelace and other research groups. This mice strain is being used in similar studies over decades and currently considered as an acceptable surrogate of irradiation model in humans.

### 3. Regulatory Compliance

The study will not be conducted in compliance with U.S. FDA 21 CFR Part 58 (Good Laboratory Practices for Nonclinical Laboratory Studies), consistent with the exploratory nature of toxicology study and is not intended to fulfill formal regulatory requirements consistent with Investigational New Drug Applications or other FDA regulatory submissions. However, the principles of the regulations will be followed including documentation and protocol and SOP adherence.

The study will be conducted in Lovelace Biomedical's animal research facilities, which are fully accredited by the Association for Assessment and Accreditation of Laboratory Animal Care (AAALAC) International. The study will comply with all applicable sections of the Final Rules of the Animal Welfare Act regulations (9 CFR Parts 1, 2, and 3, as applicable), as well as the Guide for the Care and Use of Laboratory Animals (2011). The study notebook and study binder will be quality control (QC) reviewed at the conclusion of the study.

### 4. Test Article

Test Articles (TAs) will be characterized by the Sponsor or designee. The Sponsor will ensure that documentation on the identity (suppler/manufacturer), batch number and/or lot number, uniformity, and stability for the test articles is provided for inclusion in the Final Report.

### 4.1. Test Article 1

Identity: Ruxolitinib
Description: Ruxolitinib as a phosphate salt with a correction factor of 1.32.
Storage Conditions: Room temperature for up to 1 year

### 4.2. Test Article 2

Identity: Itacitinib
Description: Itacitinib as an adipic acid salt with a correction factor of 1.21.
Storage Conditions: Room temperature for up to 1 year

### 4.3. Test Article 3

Identity: Parsaclisib
Description: Parsaclisib as a hydrochloric acid salt with a salt correction factor of 1.08.
Storage Conditions: Room temperature for up to 1 year

### 4.4. Test Article 4

Identity: Povorcitinib
Description: Povorcitinib as a phosphate salt with a correction factor of 1.32.
Storage Conditions: Room temperature for up to 1 year

### 5. Test System

Species/Strain: C57Bl/6 mice
Target Age at Arrival/Study Start: 8-10 weeks/10-12 weeks
Target Body Weight Range at Study Start: 20-35 g
Identification: All animals will be uniquely identified by microchip (BMDS), alphanumeric color-coded tail marking, cage cards, and/or shaving patterns per SOP ACG-2126, Animal Identification.
Number on Study/Sex: 110 males (plus 5 spares)
Source: Jackson Labs East
Randomization: Animals will be assigned to groups using Provantis with a stratified (body weight) randomization procedure. Animals will be placed in groups so that animals with the most similar body weights will be included on study.

### 6. Animal Husbandry

Housing: Animals will be group-housed with husbandry performed according to SOP ACS-0075, Rodent Husbandry and Manual Restraint.
Quarantine Period: The animals will be quarantined per SOP ACL-2127, Receipt and Quarantine of Animals.
Food: Envigo Certified Global Diet 2016 (Envigo, Madison, WI), restricted diet, once daily. Each batch of feed is analyzed per SOP ACG-2142, Receipt, Analysis, Storage, and Disposition of Animal Feed, Water, and Bedding, and will be used within the manufacturer's designated shelf-life. The Study Director will review the feed analyses documentation and copies of the analyses will be included in the study files.
Water: C57Bl/6 mice will receive municipal water, filtered at 5 μm, 2 μm, and 0.2 μm, unlimited access, except during irradiations. Water is analyzed at least annually per SOP ACG-2142, Receipt, Analysis, Storage, and Disposition of Animal Feed, Water, and Bedding. The Study Director will review the water analysis documentation and copies of the analyses will be included in the study records.
Environmental Conditions: The targeted conditions for animal room environment and photoperiod will be as follows:
   Temperature: 20-26° C.
   Humidity: 30-70%
   Light Cycle: 12:12 hour light: dark cycle (which may be interrupted for study activities).

Excursions from these environmental conditions that may have an impact on the study will be reviewed by the Study Director and noted in the study file. Light, humidity, and temperature excursions are defined as a sustained reading that falls out of range for more than 3 hours. Other excursions will be dealt with on a case-by-case basis.
Morbidity and Mortality: Animals will be observed twice daily for morbidity and mortality. The Study Director will be notified of animals of questionable health. If found moribund, animals will be euthanized by an overdose of an approved euthanasia solution according to SOP ACS-0334, Euthanasia of Small Animals.
Health Status: Only healthy animals will be used on study. A laboratory animal veterinarian or designee will release from quarantine per SOP ACL-2127, Receipt and Quarantine of Animals.
Palliative and/or Prophylactic Treatments: The Study Director in consultation with the Veterinary staff may approve palliative and/or prophylactic treatments under emergency situations. If needed, the animal may be removed from the study upon consultation with the Sponsor representative. In addition, the Sponsor representative will be notified of the treatment and documented in the Study file.
Enrichment: Enrichment will be provided per SOP ACG-1951, Behavioral Management and Enrichment for All Animals House at LBRI. Animals will receive, when appropriate, environmental stimulation such as, but not limited to, Plexiglas tubes, nylabones, bells, etc. Food enrichment, especially in the case of sustained inappetence, may be provided with prior approval by the Study Director.

### 7. Experimental Design

The experimental design for this study is shown in Table 1.

TABLE 1
Experimental Design

| | Treatment | Radiation | TA Dose^{C} | Dose | | |
|---|---|---|---|---|---|---|
| Group | (TA^{A}) | Exposure | (mg/kg) | Schedule | # Animals | Necropsy |
| 1 | Sham Control | No | N/A | N/A | 10 | Day 30 |
| 2 | Irradiation | Single dose | N/A | N/A | 10 | Day 30 |
| | Control | of TBI^{B} | | | | |
| 3 | Ruxolitinib | Single dose | 10.0 | BID^{D} | 10 | Day 30 |
| | (Prophylactic) | of TBI^{B} | | | | |
| 4 | Ruxolitinib | Single dose | 10.0 | BID^{D} | 10 | Day 30 |
| | | of TBI^{B} | | | | |
| 5 | Ruxolitinib | Single dose | 60.0 | BID^{D} | 10 | Day 30 |
| | | of TBI^{B} | | | | |
| 6 | Itacitinib | Single dose | 10.0 | BID^{D} | 10 | Day 30 |
| | | of TBI^{B} | | | | |
| 7 | Itacitinib | Single dose | 60.0 | BID^{D} | 10 | Day 30 |
| | | of TBI^{B} | | | | |
| 8 | Parsaclisib | Single dose | 0.3 | BID^{D} | 10 | Day 30 |
| | | of TBI^{B} | | | | |
| 9 | Parsaclisib | Single dose | 3.0 | BID^{D} | 10 | Day 30 |
| | | of TBI^{B} | | | | |
| 10 | Povorcitinib | Single dose | 3.0 | QD^{E} | 10 | Day 30 |
| | | of TBI^{B} | | | | |
| 11 | Povorcitinib | Single dose | 10.0 | QD^{E} | 10 | Day 30 |
| | | of TBI^{B} | | | | |

^{A}TA: Test Article
^{B}TBI: Total Body Irradiation, 7.0 Gy
^{C}All animals in treatment groups will be administered TA beginning Day 1. Animals in the prophylactic group will be administered TA beginning Day −2 (2 days prior to irradiation)
^{D}BID: twice/day (as “Bis In Die”, Lat.)
^{E}QD: once daily (as “Quaque Die”, Lat.)

The animals in all irradiation groups (including nine treatment groups and a radiation control group; total of 100 mice) will be exposed to a single dose (7.0 Gy) of TBI. One more group (10 animals) will be used as a Sham Control. Thus, total of 110 mice will be used in the study.

To determine the efficacy, specifically the survival benefit and progression of hematological repair of the TAs in a hARS mouse model, ten experimental groups (each ten animals) will be used: the Irradiation Control group, three Ruxolitinib groups (treated with Prophylactic, Low, and High doses, respectively), two Itacitinib groups (treated with Low and High doses, respectively), two Parsaclisib groups (treated with Low and High doses, respectively), and two Povorcitinib groups (treated with Low and High doses, respectively). Additionally, Sham Control group (ten animals) will be used as a negative control (no irradiation or medication treatment) (See Table 1).

In the end of the study, animals will be euthanized and necropsy/gross tissue harvest with major organ pathology and hematology will be performed.

### 8. Animal Identification, Conditioning, and Randomization

Animals will be uniquely identified by cage cards and subcutaneous microchips implanted after the end of the quarantine period. Additionally, tail markings may be applied as a secondary identifier.

After the end of the quarantine period, animals not excluded from study for health or other reasons will be weighed per SOP TXP-1924 Procedure for Recording Temperatures and Weighing Animals and Tissues for Studies and randomly assigned to study and exposure groups shown in. Randomization will be done by animal weight using a computerized data acquisition system (Provantis, Instem LSS Ltd., Staffordshire, UK) to normalize the average body weight of each group.

The spare animals will not be exposed to the test article; they will be transferred to another approved IACUC protocol or euthanized as described for study animals.

### 9. Irradiation

The Phillips X-ray radiation will be used for irradiation. The morning of irradiation, animals will be transported to the irradiation facility in their home cages. Prior to irradiation, animals will be anesthetized with the ketamine/xylazine cocktail (80-100 mg/kg/5-10 mg/kg, respectively). Animals will be placed into an exposure panel and irradiated per SOP TXP-1008 Operation, Maintenance, and Calibration of the Philips RT 250 X-Ray Therapy Unit. Animals will receive a single dose of TBI of 7.0 Gy. Post irradiation, the animals will be placed back in their cages and may be IM-administered, whenever is necessary, with antisedan (per veterinary discretion). The animals will be monitored for 30±15 minutes to ensure recovery from the irradiation procedures.

### 10. Supportive Care

Hydration gel (labgel Boost) will be provided to animals beginning at minimum on irradiation day and changed daily throughout the study. Additional supportive care, subcutaneous fluids (0.5 mL) will be administered if the animals are showing signs of dehydration or with a weight loss of 10% (from irradiation day).

### 11. Test Article Formulation and Dose Administration

### 11.1. Formulation

Test articles will be prepared as follows:
Ruxolitinib: reconstituted in 5% N,N-Dimethylacetamide (Sigma-Aldrich #: 270555) added to 95% of (0.5% Methyl Cellulose). The ruxolitinib is used as a phosphate salt with a correction factor of 1.32. If the dosing solution calls for 10 mg of compound, then use 13.2 mg, to account for the salt in the compound. Prepared dosing solution should be kept at room temperature and be vortexed just before each dose administering. The dry compound is stable at room temperature for up to a year.
   Example: to prepare to dose 10 mL of 10 mg/kg dose, weigh out 13.2 mg of ruxolitinib (10 mg×1.32 salt correction factor) and reconstitute in 0.5 mL dimethylacetamide, vortex until completely dissolved and then add 9.5 mL of the 0.5% methyl cellulose. Prepared dosing solution should be kept at room temperature and vortexed just before each dose.
Itacitinib: reconstituted in 5% N,N-Dimethylacetamide (Sigma-Aldrich #: 270555) added to 95% of (0.5% Methyl Cellulose). The compound should be weighed out into a polypropylene tube where the dimethylacetamide is added first to completely dissolve the compound and then the methyl cellulose is added. Itacitinib is used as a adipic salt with a correction factor of 1.21. The suspension is appeared to be cloudy, but should be uniform thereafter. The prepared dosing solution should be kept at room temp and mixed just before each dose administering. Dosing solution is stable for 3 days at room temperature. The dry compound is stable at room temperature for up to a year.
   Example: to prepare 1.5 mL to dose 5 mice with 50 mg/kg, weigh out 9.1 mg of itacitinib (7.5 mg×1.21 correction factor) and reconstitute in 0.075 mL dimethylacetamide, vortex until completely dissolved and then add 1.425 mL of the 0.5% methyl cellulose. The suspension will be cloudy, but should be uniform. Keep the prepared dosing solution at room temperature and mix just before each dose.
Parsaclisib: reconstituted in 5% N,N-Dimethylacetamide (Sigma-Aldrich #: 270555) added to 95% of (0.5% Methyl Cellulose). The compound can be weighed out into a polypropylene tube where the dimethylacetamide is added first to completely dissolve the compound and then the methyl cellulose is added. Dosing solution is stable for 3 days at room temperature. Dry compound is stable at room temperature for up to a year.
   Example: to prepare to dose 10 mL of 0.5 mg/kg dose, weigh out 0.54 mg of parsaclisib (0.5 mg×1.08 salt correction factor) and reconstitute in 0.5 mL dimethylacetamide, vortex until completely dissolved and then add 9.5 mL of the 0.5% methyl cellulose. Keep the prepared dosing solution at room temperature and mix just before each dose.
Povorcitinib: reconstituted in 5% N,N-Dimethylacetamide (Sigma-Aldrich #: 270555) added to 95% of (0.5% Methyl Cellulose). The compound can be weighed out into a polypropylene tube where the dimethylacetamide is added first to completely dissolve the compound and then the methyl cellulose is added. The addition of a couple 4-6 mm glass beads to tube aids in the solubilization of the compound. Povorcitinib is used as a phosphate salt with a correction factor of 1.32. If the solution calls for 10 mg of compound then use 13.2 mg, (10 mg×1.32 salt correction factor) to account for the salt in the compound. Dosing solution is stable for 3 days at room temperature. The dry compound is stable at room temperature for up to a year.
   Example: to prepare to dose of 10 mg/kg, weigh out 1.32 mg of povorcitinib and reconstitute in 0.1 mL dimethylacetamide, add the glass beads and vortex until completely dissolved and then add 1.9 mL of the 0.5% methyl cellulose. Keep the prepared dosing solution at room temperature on a wheel or shaker and mix just before each dose.

### 11.2 Administration

Animals will be administered test article, 10 mL/kg, as assigned in Table 1 per SOP TXP-0671 Gavage and Oral Dosing of Rodents. For animals scheduled for twice daily dosing, all efforts will be made to have a minimum of 7±1 hours between dosing sessions.

### 12. Observations and Measurements

### 12.1. Data Collection

General Data Documentation Note: Whenever possible, a validated computerized data acquisition system [e.g., Provantis™ or Animal Management System (AMS, Lovelace Biomedical, Albuquerque, NM)] will be used for data acquisition, recording, and or analysis. If unavailable, data will be recorded on pre-prepared forms for each animal, and then entered retrospectively to the electronic system. Hard copy forms will be prepared as backup to Provantis or AMS and will be used when electronic data collection is not feasible. Excursions in refrigerators and freezers temperature may be expected due to use; excursions per FCP-1172 Monitoring of Refrigerators, Freezers, and Ultra-lows will be assessed for study impact by Study Director or designee.

### 12.2 Clinical Observations

Animals will be examined minimum of twice daily (morning and afternoon) from arrival to the day of necropsy by animal resources/laboratory animal sciences personnel per SOP ACS-0075 Rodent Husbandry and Manual Restraint and documented in AMS. Examinations will also be oriented toward (1) identifying dead, weak, or moribund animals, and (2) documenting the onset and progression of any abnormal clinical signs. The criteria evaluated for defining an animal as moribund include anorexia, dehydration, hunched posture, reluctance to move, lateral recumbency, and heavy breathing. The Study Director will make decisions regarding the euthanasia of weak or moribund animals after consultation with a staff veterinarian. Moribund or dead animals will be necropsied as soon as possible after being found but in no case later than 16 hours after being found. In the event that the animal carcass needs to be stored it will be refrigerated (not frozen).

### 12.3 Detailed Observations and Euthanasia Criteria

Detailed clinical observations will be performed twice a day (a.m. and p.m.), with special attention to normal behavior per SOP TXP-1532, Pharmacologic and Toxicologic Observations of Experimental Animals starting 2 days prior to the day of irradiation (i.e., Day “−2”) and continuing until the end of the study, or until moribund or found dead. Additional observations may be made added at the discretion of the Study Director. Should the animals experience acute test article toxicity, suffer some unforeseen injury while on-study or if the animals are showing signs including but not limited to: abnormal appearance (piloerection), lethargy, changes in respiratory pattern, neurological signs, abnormal behaviors, cyanotic discoloration of mucous membranes, irregular discharge from orifices or bloody stool/urine, then the Study Director and veterinary staff will be consulted for an evaluation of the animal's well-being. Special attention will be paid to clinical signs include but are not limited to apnea, dyspnea (labored breathing), malaise, marked nasal discharge, abnormal heartbeat, cyanosis, discoloration of mucous membrane, excessive body weight loss, etc. Animals showing severe signs of distress will be euthanized immediately at the discretion of the Study Director in consultation with veterinary staff. Established warrant euthanasia criteria will be: >20% body weight loss from baseline, lethargy, and/or reluctance to move. Any one of the criteria will trigger euthanasia.

### 12.4. Body Weights

All animals will be weighed after release from quarantine and that weight will be the pre-study body weight used to randomize animals into dose groups per TXP-1924 Procedure for Recording Temperatures and Weighing Animals and Tissues for Studies. Throughout the duration of the study, body weights will be collected a minimum of three times per week post irradiation. All assigned study animals also will be weighed at necropsy. Additional weights may be collected at the discretion of the Study Director.

### 12.5. Blood Collections

At necropsy, blood will be collected via per SOP ACS-1287 Procedures for Injections, Blood Withdrawal and Dermal Dosing and Blood Withdrawal in Rodents.

### 12.6. Hematology

Blood samples for hematology analyses collected during necropsy will be transferred to the Lovelace Biomedical Clinical Pathology department for analysis per SOP PAC-0736, Collection and Processing of Samples for Clinical Pathology.

For hematology analyses, whole blood will be collected and placed into tubes containing ethylene diamine tetra acetate (K3EDTA or K2EDTA) as an anticoagulant. Hematology samples will be analyzed on an ADVIA™ 2120 Hematology System (Siemens Medical Solutions Diagnostics, Tarrytown, NY) per SOP PAC-2121 ADVIA™ 2120 Hematology Analyzer. The hematology parameters to be measured or calculated are shown in Table 2. Any remaining blood not used for analysis will be discarded.

Evaluations will be performed on all study animals for which adequate sample volumes are obtained and for which no analytical problems are encountered. Remaining blood will be saved for possible future analysis. If target collection volumes are not obtained or if evaluations are not performed, a reason and notation will be included in the data.

TABLE 2
Hematology Parameters

| Parameter | Abbreviation^{A} | Units |
|---|---|---|
| Red Blood Cell Count | RBC | 106/μL |
| Hemoglobin | HGB | g/dL |
| Hematocrit | HCT | % |
| Mean Corpuscular Volume | MCV | fL |
| Mean Corpuscular Hemoglobin | MCHC | g/dL |
| Concentration | | |
| Mean Corpuscular Hemoglobin | MCH | pg |
| Red Cell Distribution Width | RDW | % |
| Platelet Count | PLT | 103/μL |
| Mean Platelet Volume | MPV | fL |
| Platelet Distribution Width | PDW | % |
| Reticulocytes (Absolute) | RETIC | 109/L |
| Percent Reticulocytes (Relative) | RETIC | % RBC |

White Blood Cell Count and Absolute and Relative Differential

| | | |
|---|---|---|
| White Blood Cell Count | WBC | 103/μL |
| Neutrophils | PMN | 103/μL (and %) |
| Lymphocytes | LYM | 103/μL (and %) |
| Monocytes | MONO | 103/μL (and %) |
| Eosinophils | EOS | 103/μL (and %) |
| Basophils | BASO | 103/μL (and %) |
| Large Unstained Cells | LUC | 103/μL (and %) |

^{A}The final results may be reported as described above or using the terms/abbreviations from the analyzer as appropriate.

### 13. Pathology

At scheduled necropsy or in cases of morbidity, animals will be euthanized by intraperitoneal injection of an overdose of a barbiturate-based euthanasia solution following SOP ACS 0334 Euthanasia of Small Animals. Detailed gross necropsies will be performed on all animals (found dead, moribund euthanasia, or scheduled euthanasia) per SOP PAN 0455 Small Animal Necropsy Procedure and will consist of a complete external and internal examination including cranial, thoracic, and abdominal organs and tissues. All gross findings will be recorded in descriptive terms, which may include morphology, location(s), severity, size, shape, color, consistency, and number. Photographs may be used to document unusual or characteristic lesions, per SOP PAN 1892 Operation and Maintenance of the Life Sciences Digital Cameras if applicable. Complete details will be included in the final report.

Blood will be collected, when possible, at scheduled and unscheduled necropsy as described below for various analyses, and organs collected, weighed, and preserved for possible histopathology (Table 3). Gross lesions or other additional tissues which may help explain clinical signs or the pathogenesis of findings may be preserved for possible histopathological evaluation (including tissues from control or other animals which may serve as reference tissues for findings in some study animals).

Found dead or moribund animals will receive the study protocol specified tissue collection. No blood will be collected for animals found dead or that are moribund. Animals found dead will be refrigerated until a necropsy can be performed, and will have the full protocol set of tissues collected without organ weights. A cause of death will be determined if possible based on gross findings.

TABLE 3
Routine Organs/Tissues

| Tissues | Weigh# | Comments* |
|---|---|---|
| Abnormal Tissue | | Representative examples. |
| | | Collection and histologic |
| | | examination of redundant/related |
| | | gross observations or those with |
| | | an obvious gross diagnosis will |
| | | be at the discretion of the |
| | | pathologist and/or SD. |
| Aorta (thoracic) | | |
| Bone marrow smear | | |
| Brain | X | |
| Cervix (Female) | | |
| Epididymis (Male) × 2 | X | Fixed in Modified Davidson's |
| | | Fluid. |
| Esophagus | | |
| Eye × 2 (+Optic Nerve) | | Fixed in Modified Davidson's |
| | | Fluid. At least one optic nerve |
| | | should be present on the slide. |
| Femur (with bone marrow) | | Fix left; bone marrow smear |
| | | from right. |
| Gastrointestinal tract: | | |
| Stomach Duodenum Jejunum | | |
| Ileum Cecum Colon Rectum | | |
| Heart | X | |
| ID (microchip/tail ID) | | Retained for identification |
| | | purposes. |
| Kidney × 2 | X | |
| Liver | X | Samples from two lobes. |
| Lungs | X | |
| Lymph Nodes | | Mandibular, mesenteric. |
| Mammary Gland (inguinal) | | Collected with skin. |
| Ovary (Female) × 2 | X | |
| Pancreas | | |
| Parathyroid × 2 | | |
| Pituitary | | |
| Sciatic Nerve | | |
| Salivary Gland | | Mandibular |
| Skin | | Collected with mammary gland. |
| Skeletal Muscle | | Thigh |
| Spinal Cord | | Cervical, midthoracic and |
| | | lumbar |
| Spleen | X | |
| Sternum (including Bone | | |
| Marrow) | | |
| Testis (Male) × 2 | X | Fixed in Modified Davidson's |
| | | Fluid. |
| Thymus | X | |
| Thyroid × 2 | | |
| Urinary Bladder | | |
| Uterus (Female) | X | Body and horns |
| Vagina (Female) | | |

*All tissues not listed as” or “Fixed in Modified Davidson's” are fixed in 10% neutral buffered formalin.
#Weighed paired organs together.

Representative samples of the tissues listed in Table 3 will be harvested from all animals and fixed in 10% neutral buffered formalin (NBF). Other tissues may be preserved at the discretion of the Study Director and/or Study Pathologist. The Study Director, their designee, and/or Pathologist will determine the extent to which autolysis may affect tissue preservation and analyses on those animals found dead. All carcasses will be discarded following tissue sampling.

All samples will be held for up to 1 year. If a decision is made regarding analysis, the protocol will be revised. If no decision is made, the sponsor will be contacted for disposal and shipment of samples.

### 14. Records to be Maintained

Specimens shall be identified by test system, study, nature, and date of collection.

All raw data and records that would be required to reconstruct the study will be maintained at Lovelace for a minimum of one year.

Records retained shall include:
Test Materials:
   Test material receipt storage, usage, and disposition
   Dosing and formulation data
Facility:
   Animal room temperature and humidity
   Water analyses
   Feed identification, analyses, and usage
   Animal care records
   Medical records
In-Life Phase:
   Animal receipt and disposition
   Quarantine observation, health assessment, and release
   Animal randomization
   Body weights
   Treatment (if applicable)
   Animal observations
   Sample collection
Clinical Pathology:
   Sample collection and processing data

### Example B: Preliminary Survival Data

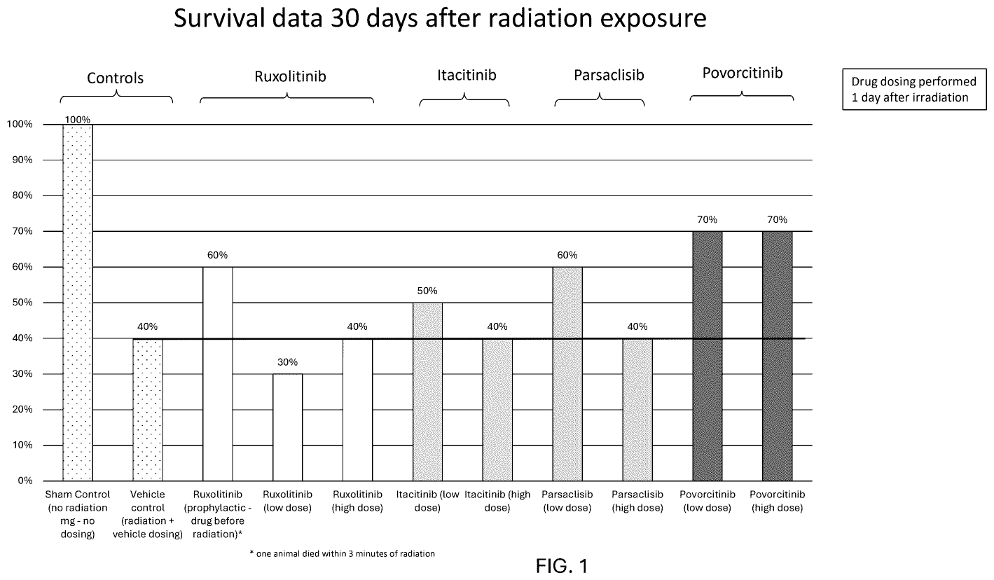

FIG. 1 shows preliminary survival data 30 days after radiation exposure of C57Bl/6 mice treated with ruxolitinib, itacitinib, parsaclisib, or povorcitinib with the protocol outlined in Example A.

Mice treated with prophylactic ruxolitinib, low dose itacitinib, low dose parsaclisib, and both low and high dose povorcitinib demonstrated improved survival rates compared with the vehicle control mice.

### Example C: Bone Marrow Recovery Preservation

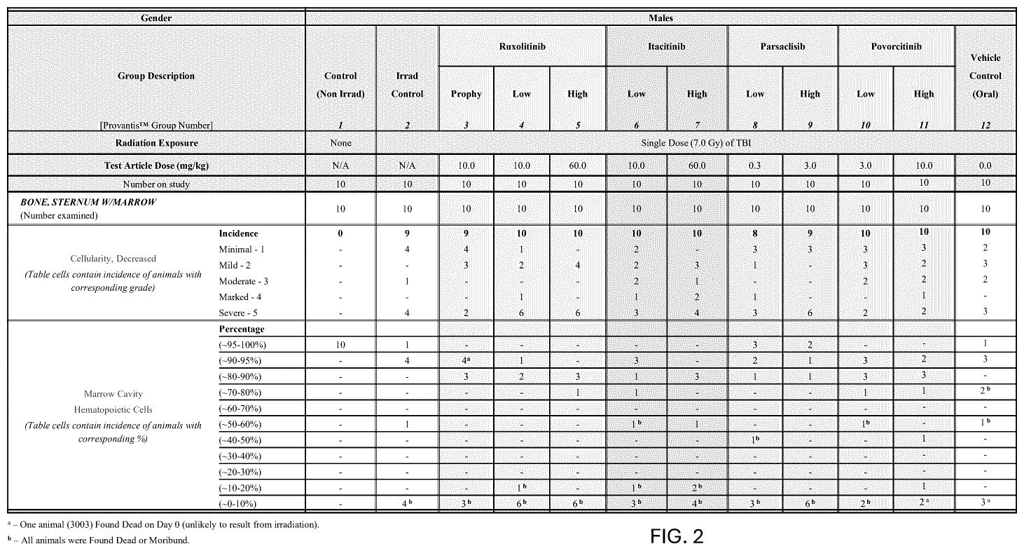

FIG. 2 shows bone marrow recovery preservation data 30 days after radiation exposure of C57Bl/6 mice treated with ruxolitinib, itacitinib, parsaclisib, or povorcitinib with the protocol outlined in Example A.

Administration of ruxolitinib, itacitinib, parsaclisib, or povorcitinib following radiation exposure resulted in evidence of bone marrow repopulation, supporting a role in hematopoietic recovery.

### Example D: Kaplan Mier Graph

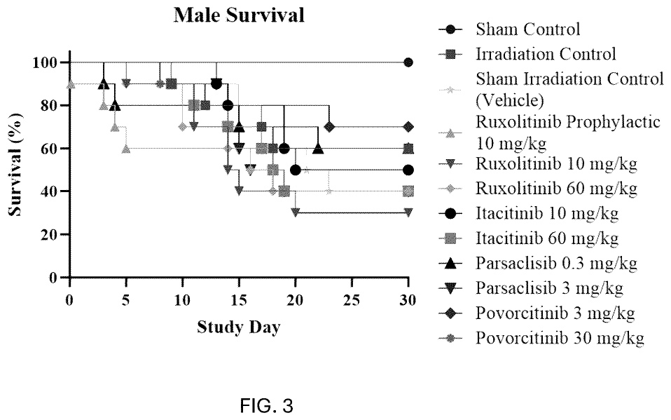

FIG. 3 shows male survival data 30 days after radiation exposure of C57Bl/6 mice treated with ruxolitinib, itacitinib, parsaclisib, or povorcitinib with the protocol outlined in Example A.

Administration of ruxolitinib, itacitinib, parsaclisib, or povorcitinib following radiation exposure generally resulted in similar or improved survival rates to vehicle.

Various modifications of the invention, in addition to those described herein, will be apparent to those skilled in the art from the foregoing description. Such modifications are also intended to fall within the scope of the appended claims. Each reference cited in the present application, including all patent, patent applications, and publications, is incorporated herein by reference in its entirety.

1. A method of treating or preventing a disease or disorder related to radiation exposure in a patient in need thereof, comprising administering to the patient a therapeutically effective amount of a compound or a pharmaceutically acceptable salt thereof which inhibits JAK1, JAK1 and JAK2, or PI3Kδ.

2. The method of claim 1, wherein the compound or pharmaceutically acceptable salt thereof inhibits JAK1 and JAK2, and is selective for JAK1 and JAK2 over JAK3 and TYK2.

3. The method of claim 2, wherein the compound or pharmaceutically acceptable salt thereof is ruxolitinib, deuterated forms thereof, stereoisomers thereof, and/or pharmaceutically acceptable salts thereof.

4.-9. (canceled)

10. The method of claim 1, wherein the compound or pharmaceutically acceptable salt thereof inhibits JAK1, and is selective for JAK1 over JAK2, JAK3, and TYK2.

11. The method of claim 10, wherein the compound or pharmaceutically acceptable salt thereof is itacitinib, deuterated forms thereof, stereoisomers thereof, and/or pharmaceutically acceptable salts thereof.

12.-17. (canceled)

18. The method of claim 10, wherein the compound or pharmaceutically acceptable salt thereof is povorcitinib, deuterated forms thereof, stereoisomers thereof, and/or pharmaceutically acceptable salts thereof.

19.-24. (canceled)

25. The method of claim 1, wherein the compound or pharmaceutically acceptable salt thereof inhibits PI3Kδ, and is selective for PI3Kδ over PI3Kα, PI3Kβ, and PI3Kγ.

26. The method of claim 25, wherein the compound or pharmaceutically acceptable salt thereof is parsaclisib, deuterated forms thereof, stereoisomers thereof, and/or pharmaceutically acceptable salts thereof.

27.-33. (canceled)

34. The method of claim 1, wherein the radiation exposure is via non-therapeutic radiation.

35. The method of claim 1, wherein the disease or disorder is acute radiation syndrome.

36.-44. (canceled)

45. The method of claim 1, wherein the radiation exposure is via therapeutic radiation.

46.-59. (canceled)

60. The method of claim 1, wherein the compound or pharmaceutically acceptable salt thereof is administered prior to exposure of the patient to radiation.

61.-62. (canceled)

63. The method of claim 1, wherein the compound or pharmaceutically acceptable salt thereof is administered after exposure of the patient to radiation.

64.-70. (canceled)

71. The method of claim 1, wherein the compound or pharmaceutically acceptable salt thereof is administered to the patient in a pharmaceutical composition comprising the compound and a pharmaceutically acceptable carrier or excipient.

72.-74. (canceled)

75. The method of claim 71, wherein the administration is pulmonary.

76. (canceled)

77. The method of claim 71, wherein the administration is parenteral.

78. The method of claim 77, wherein the parenteral administration is an intramuscular or subcutaneous injection.

79. The method of claim 77, wherein the parenteral administration is in the form of a long acting depot formulation.

80. The method of claim 71, wherein the administration is oral.

81.-82. (canceled)

83. The method of claim 71, wherein the administration is topical.

84.-85. (canceled)

86. The method of claim 1, further comprising administering one or more additional therapeutic agents.

87.-107. (canceled)

108. A method of increasing the survival rate of a patient exposed to radiation, comprising administering to the patient a therapeutically effective amount of a compound or a pharmaceutically acceptable salt thereof which inhibits JAK1, JAK1 and JAK2, or PI3Kδ.

109.-120. (canceled)

121. A kit comprising: (a) ruxolitinib, povorcitinib, itacitinib, parsaclisib, or a pharmaceutically acceptable salt of any of the aforementioned; and (b) instructions for using the ruxolitinib, povorcitinib, itacitinib, parsaclisib, or a pharmaceutically acceptable salt of any of the aforementioned for treating or preventing acute radiation syndrome of a patient.

122.-123. (canceled)
